(19) United States
(12) Patent Application Publication
(10) Pub. No.: US 2005/0282196 A1
(43) Pub. Date: Dec. 22, 2005

(54) Methods and compositions for cancer diagnosis

(76) Inventor: Jose Costa, Guilford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/120,435

(22) Filed: May 2, 2005

Related U.S. Application Data
(60) Provisional application No. 60/567,161, filed on Apr. 30, 2004.
(60) Provisional application No. 60/645,148, filed on Jan. 19, 2005.

(51) Int. Cl. C12Q 1/68

(52) U.S. Cl. 435/6

(57) ABSTRACT

This invention relates generally to the field of cancer diagnostics. The invention further relates to the use of mutational load distribution analysis (MLDA) to examine changes in the distribution of genetic mutations incident to cancer.

### FIELD OF THE INVENTION

This invention relates generally to the field of cancer diagnostics. The invention further relates to the use of mutational load distribution analysis (MLDA) to examine changes in the distribution of genetic mutations incident to cancer.

### BACKGROUND OF THE INVENTION

For most organisms, the process of carcinogenesis encompasses relatively long periods of time and for the common epithelial tumors of the human, the period of tumor development spans a decade or more. (Bhatia et al. J of Clin Onc 2003: Vol 21, No 23 (December 1); 4386-4394.) Tissues are constantly under the assault of environmental mutagens but damage is largely neutralized by DNA repair mechanisms. (Rouse et al. Science 2002: Vol. 297(5581); 547-51.) Studies of non-neoplastic tissues in asymptomatic individuals that are unlikely to develop tumors show the presence of mutations (Dolle et al. Nature Genetics 1997: Vol 17; 431-434; King et al. Mutation Research 1994: Vol 316; 79-90.) and even when occurring in cancer genes, mutations are cleansed from the cellular constituents of a tissue. This constant low level of mutation and cleansing, produces a random fluctuation of mutations that affects the cellular composition of a tissue albeit in a very low proportion of cells.

Under physiological conditions, the structural and functional integrity of tissues is insured by a compartmental organization (Mintz. Symp Soc Exp Biol 1971; Vol 25: 345-370.) and spatial constraints regulate the co-existence of physiological clonal patches maintained by stem cells. Each patch is populated by the replication of symmetrically dividing daughters of the stem cell that subsequently differentiate and eventually engage the apoptotic program. The introduction of mutation and aneuploidy in tissue stem cells (Cairns. Nature 1975; Vol 255:197-200, Cairns. Proc Natl Acad Sci USA 99 2002; 10567-10570.) alters the ecology of the clonal cell populations that compose a tissue and create a collection of subpopulations of the same cell type occupying separate patches of a subdivided habitat (metapopulations). The widely accepted ecological concept that disturbances (exogenous agents of mortality) have pronounced effects on diversity (Rainey et al. TREE 2000; Vol 15(6): 243-247, Buckling et al. Nature 2000 (Dec. 21-28); Vol 408(6815): 961-4.) suggests that repeated insults that affect tissues are likely to influence the metapopulation dynamics of the clonal patches composing them. Under these circumstances, the three conditions necessary for an evolutionary process to occur, variation (mutation, epigenetic alterations), competition (differential fitness) and replication are met and henceforth carcinogenesis can be regarded as a micro-evolutionary process acting on a metapopulation of cells. During carcinogenesis it is the complex phenotype of the tumor stem-cell that is the target of selection. Mutation, drift and selection are the forces that underlie the exploration of the phenotypic space for the complex set of traits that characterize tumor cell populations. It is the combination of mutations and epigenetic changes occurring in a small subset of several hundred cancer genes that leads to the emergence of the complex cellular behavior that characterizes malignant tumors. (Hahn et al. Nat Rev Cancer 2002; 2: 331-41.) Although there is considerable variation, common tumor types are defined by a limited set of genetic alterations (cf. CGAP) that represent the most frequent final states of an evolutionary process for which we remain largely ignorant about the exact genealogy.

Colorectal cancer remains the second leading cause of cancer in Western countries and accounts for more than 10% of all cancer deaths. Its progressive nature—adenoma followed by carcinoma is believed to occur in most patients—and accessibility by non-surgical methods makes it suitable for early detection and prevention. Most adenomas are treated successfully by endoscopic polypectomy and survival rates for patients with tumors diagnosed at early stages are better than after lymph node dissemination has occurred.

Pancreatic cancer (or cancer of the pancreas) is the fifth leading cause of cancer death in the United States; approximately 28,000 Americans die annually from pancreatic cancer. This cancer is considered extremely difficult to treat. Further, surgical removal (“resection”) of the cancer by a “pancreaticoduodenectomy” or “Whipple procedure” is currently the only current treatment for patients with pancreatic cancer.

The factors that guide the evolution of a tumor share many similarities with macroevolution (Bodmer W. and Tomlinson I. Nature Medicine 5:11-2,1999). During the earliest phases of the process, micro-clones of cells harboring mutations in genes implicated in the pathogenesis of tumors can be found to co-exist in tissues at risk for carcinoma (Moskaluk, Calif., et al., Cancer Research, 57:2140-43,1997; Deng, G, et al., Science 274:2057-59,1996; Chaubert P, et al., Am. J. Pathology 144:767-75,1994). Mutated alleles spread first within the clonal patches that constitute the developmentally regulated units of tissue architecture. For example, in the colon the physiologic deme is the crypt. Under normal circumstances, mutations accumulate randomly in each deme. When these mutations lead to favored growth of a single deme, yielding an oncodeme, the overall mutational complexity of the tissue is reduced. These changes may be impaired by morphologic criteria. As indicated above, when a clone harbors a mutation in a gene implicated in the pathogenesis of cancer, it can be designated as an oncodeme. Increased risk of cancer has been correlated with certain diseases (precancerous conditions, e.g. atrophic gastritis) or to morphological alterations known as preneoplastic lesions (low, moderate and severe dysplasia). Extensive studies in epithelial organs have suggested that there is a dysplasia-to-carcinoma sequence representing the morphological manifestation of the emergence of a neoplasm. Yet, molecular genetic studies of coexisting early carcinoma and dysplastic lesions in tissues at risk for cancer suggest that diversity can be found among dysplastic lesions located in the vicinity of a tumor, and that a direct linkage between dysplasia and carcinoma is not easily demonstrated (Lin M C, et al., Am. J. Pathology 152:1313-8,1998). Complete replacement of the precursor lesion by microinvasive carcinoma may in part explain this difficulty. However, a surprising finding of these studies is the demonstration of mutated cancer genes in lesions not known to carry an elevated risk of transformation, and even in morphologically normal tissues in the vicinity of a carcinoma. Thus, molecular preneoplasia does not have a necessary morphological correlate.

A diversity of mutations, both in terms of the genes affected and the mutated alleles, can be found in tissues known to be at high risk for carcinoma or already bearing a tumor. At least in two experimental rat models, N-methyl-nitrosourea (NMU) induced mammary carcinomas (Cha E. S., et al., Carcinogenesis 17:2519-24,1996) and azoxymethane (AOM) related colonic carcinomas, mutations in the ras family of oncogenes occur in the absence of chemical mutagenesis. These results are of particular interest because at least some of the same mutated ras alleles can be found in the tumor, indicating they have been selected for during tumor formation.

A challenge in developing methods for early cancer evaluation is to detect the emergence of significant mutations against a background of normal mutational complexity. U.S. Pat. No. 6,428,964 discloses methods for detecting an alteration in a target nucleic acid in a biological sample. According to the invention, a series of nucleic acid probes complementary to a contiguous region of wild type target DNA are exposed to a sample suspected to contain the target. Probes are designed to hybridize to the target in a contiguous manner to form a duplex comprising the target and the contiguous probes “tiled” along the target. If a mutation or other alteration exists in the target, contiguous tiling will be interrupted, producing regions of single-stranded target in which no duplex exists. Identification of one or more single-stranded regions in the target is indicative of a mutation or other alteration in the target that prevented probe hybridization in that region.

U.S. Pat. No. 6,300,077 discloses methods for enumerating (i.e., counting) the number of molecules of one or more nucleic acid variant present in a sample. According to methods of the invention, a disease-associated variant at, for example, a single nucleotide polymorphic locus is determined by enumerating the number of a nucleic acid in a first sample and determining if there is a statistically-significant difference between that number and the number of the same nucleotide in a second sample. A statistically-significant difference between the number of a nucleic acid expected to be at a single-base locus in a healthy individual and the number determined to be in a sample obtained from a patient is clinically indicative.

U.S. Pat. No. 6,214,558 discloses methods for detecting in a tissue or body fluid sample, a statistically-significant variation in fetal chromosome number or composition to reliably detect a fetal chromosomal aberration in a chorionic villus sample, amniotic fluid sample, maternal blood sample, or other tissue or body fluid.

U.S. Pat. No. 6,203,993 discloses methods for comparing the number of one or more specific single-base polymorphic variants contained in a sample of pooled genomic DNA obtained from healthy members of an organism population and an enumerated number of one or more variants contained in a sample of pooled genomic DNA obtained from diseased members of the population to determine whether any difference between the two numbers is statistically significant. The presence of a statistically-significant difference between the reference number and the target number is indicative that the loci (or one or more of the variants) is a diagnostic marker for the disease. In a patient having a specific variant which is indicative of the presence of a disease-related gene, the severity of the disease can be assessed by determining the number of molecules of the variant present in a standardized DNA sample and applying a statistical relationship to the number. The statistical relationship is determined by correlating the number of a disease-associated polymorphic variant with the number of the variant expected to occur at a given severity level.

U.S. Pat. No. 6,143,529 discloses methods for detecting cancer or precancer by determining the amount of DNA greater than about 200 bp in length from a sick patient sample, and comparing the amount to the amount of DNA greater than about 200 bp in length expected to be present in a sample obtained from a healthy patient. A statistically significant larger amount of nucleic acids greater than about 200 bp in length in the patient sample is indicative of a positive screen.

All the above cancer detection methods are directed to detecting the presence or absence of mutated alleles, and developing a statistical correlation between the detected mutated alleles and the occurrence of cancer. However, strategies designed to simply detect the presence or absence of mutated alleles, even for genes of proven etiologic importance to cancer, most often fail to meaningfully discriminate patients with true premalignant lesions (i.e., ones that warrant therapy or increased surveillance) from patients with similar somatic changes who will never develop cancer. The reasons for this are manifold, relating primarily to the balance of host and environmental factors that modify the evolution of the clone that will become a given patient's cancer. Thus, there is a need in the art for early-detection strategies that will identify the presence of genetic changes in a tissue or tissue surrogate and detect, even against a constantly changing checkerboard of background mutations, the early emergence of a premalignant clone that is likely to progress. Moreover, there is a need for strategies to differentiate the stage of cancer to which the premalignant clone is likely to progress.

### SUMMARY OF THE INVENTION

The present invention provides novel diagnostic and therapeutic methods for use in mammalian subjects suffering from cancer. A hallmark of cancer is the modification of the genome; such modifications are broadly termed “mutations.” In particular, it has been found that the frequency of mutated alleles in a sample from the subject, or the total mutational load of the sample, are useful in predicting or determining the stage of the subject's cancer.

In one aspect of the invention, the invention provides a method of evaluating the risk of cancer development in a subject by providing from the subject a test sample of material for which the risk of cancer development is to be evaluated, quantitating the frequency of one or more mutated alleles in the test sample relative to one or more nonmutated alleles, and comparing the frequency of the one or more mutated alleles in the test sample with a reference frequency. Generally, a higher frequency of the one or more mutated alleles in the test sample than in the reference frequency indicates that the subject has an elevated risk of cancer.

The cancer may be adenoma, carcinoma in situ, or invasive carcinoma. The cancer may be present in any tissue or organ of the subject or may be present in more than one tissue or organ, and may contain a primary tumor and/or one or more metastases. For example, the cancer may be colorectal cancer or pancreatic cancer.

In embodiments of the invention, the mutated alleles are obtained from any cancer-associated gene, such as K-ras, p53, or APC. The mutated allele may be present in exon 1 of K-ras, in exon 5 of p53, or in exon 7 of p53. Specific alleles include alleles of K-ras and of p53 listed in Table 1. In embodiments of this invention, the reference frequency is derived from one or more reference subjects that do not have cancer.

Generally, any test sample from which an identification of alleles of interest can be made is provided by the present invention. Suitable test samples include blood, serum, circulating tumor cells, urine, a tumor biopsy, a tumor aspirate, a cultured tumor cell, bone marrow, a stool sample, and a colonic brushing.

In another aspect, the invention provides a method of evaluating the risk of colorectal cancer development in a subject by providing from the subject a test sample of material for which the risk of colorectal cancer development is to be evaluated, quantitating the frequency of one or more mutated alleles in the test sample relative to one or more nonmutated alleles and comparing the frequency of the one or more mutated alleles in the test sample with a reference frequency. Generally, a higher frequency of the one or more mutated alleles in the test sample than in the reference frequency indicates that the subject has an elevated risk of colorectal cancer. The colorectal cancer may be at any given stage, including adenoma, carcinoma in situ and invasive carcinoma.

The test sample may be a colonic lavage, a stool sample, or a colonic brushing. In embodiments of the invention, the test sample includes an exfoliated cell.

In another aspect, the invention provides a method of evaluating the stage of cancer development in a subject by providing from the subject a test sample of material for which the stage of cancer development is to be evaluated, quantitating the frequency of one or more mutated alleles in the test sample, and comparing the frequency of one or more mutated alleles in the test sample with the frequency of one or more reference alleles (such as an allele obtained from a subject without cancer), wherein a mutated allele in higher frequency than a reference allele indicates that the subject has a colorectal cancer of a given stage. The mutated alleles can be from genes including K-ras, p53, APC, and BAT26. Specific alleles include alleles of K-ras and of p53 as listed in Table 1. The frequency of the mutated alleles in the test sample will vary based on the cancer stage of the subject, or the subject's predisposition to cancer of a given stage. With colorectal cancer, for example, the cancer may be an adenoma, carcinoma in situ or invasive carcinoma, and the frequency of a tested allele will vary based on these stages of cancer. If the frequency of a given allele is below about 1.2% (e.g., 0.8%, 0.9%, 1.0% or 1.1%), the subject does not have colorectal cancer. If the frequency of a given allele is between about 1.0% (e.g., 1.2%) and about 9.5% (e.g., 9.0% to 10%) the subject has adenoma or carcinoma in situ, or a predisposition thereto. If the frequency of a given allele is above about 9.5% (e.g. 9.7%), the subject has invasive carcinoma, or a predisposition thereto. With pancreatic cancer, the stages are normal, precancerous pancreatitis, and pancreatic cancer. The frequency of a tested allele will vary based on these stages. If the frequency of a given allele is below about 1.2% (e.g. 1.1%, 1%, 0.9%) the subject does not have pancreatic cancer. If the frequency of a given allele is between about and about 1.0% (e.g. 1.2%, 1.3%, 1.4%) and about 4% (3.7, 3.8, 3.9, 4.0, 4.1) the subject has precancerous pancreatitis or a predisposition thereto. If the frequency of a given allele is above about 3.8% (4.0, 4.1), the subject has pancreatic cancer, or a predisposition thereto.

The step of quantitating the frequency of one or more mutated alleles in the test sample is performed using a oligonucleotide array. Alternatively, the step of quantitating includes the enhancement of a signal using rolling circle amplification.

In another aspect, the invention provides a method of diagnosis of any cancer in a subject, including colorectal and pancreatic cancer, by providing from the subject a test sample of material that contains one or more cells or cellular material, determining the frequency of mutated alleles of one or more genes (e.g., K-ras, p53, APC, and/or BAT26) in the test sample, quantitating the mutational load in the test sample, and comparing the mutational load in the test sample with a reference mutational load. Generally, quantitating the mutational load of a test sample includes determining the sum of the frequencies of specific mutated alleles. Generally, a higher mutational load in the test sample than in the reference frequency indicates that the subject has cancer. Moreover, the mutational load in the sample also provides information regarding the stage of cancer, if any, in the subject.

For example, when a total mutational load of the test sample is below about 6.2%, the subject does not have colorectal cancer. When a total mutational load of the test sample is between about 16.5% and about 22.2%, the subject has adenoma. Further, when a total mutational load of the test sample is between about 22.3% and about 36.3%, the subject has carcinoma in situ. When a total mutational load of the test sample is above about 25.1%, the subject has subject has invasive carcinoma.

In a further aspect, the invention provides a method of evaluating the likelihood of relapse of cancer in a subject having cancer following a cancer treatment, by providing from the subject a first sample and a second sample of material, wherein the second sample is provided from the subject a sufficient period of time after the first sample, quantitating the frequency of one or more mutated alleles (such as alleles of genes including K-ras, p53, APC, and BAT26) relative to one or more nonmutated alleles in the first and second samples, and comparing the frequency of the first sample with the frequency from the second sample. Generally, a higher frequency in the second sample than in the first sample indicates that the subject has an elevated risk of relapse of cancer. In embodiments of the invention, the cancer is a colorectal cancer such as adenoma, carcinoma in situ or invasive carcinoma. When evaluating the likelihood of relapse of cancer in a subject having colorectal cancer following a cancer treatment, the samples obtained can be a colonic lavage, a stool sample, or a colonic brushing.

In another aspect, the invention provides a method of determining the predisposition to a relapsing cancer of a given stage in a subject by providing from the subject a first sample and a second sample of material, wherein the second sample is provided from the subject a sufficient period of time after the first sample, quantitating the frequency of one or more mutated alleles in the first and second samples, and comparing the frequency of one or more mutated alleles in the first sample with the frequency of one or more alleles in the second sample. Generally, a mutated allele in higher frequency in the first sample than the allele in the second sample indicates that the subject is predisposed to a relapsing cancer of a given stage. The subject's cancer may be colorectal, pancreatic or any other type of cancer, and if it is colorectal cancer, it may be adenoma, carcinoma in situ or invasive carcinoma.

Generally, when the frequency of the allele in the second sample is below about 1.2%, the subject does not have relapsing colorectal cancer. When the frequency of the allele in the second sample is between about 1.2% and about 9.5%, the subject has adenoma or carcinoma in situ. When the frequency of the allele in the second sample is above about 9.5%, the subject has invasive carcinoma.

### BRIEF DESCRIPTION OF THE DRAWINGS

FIGS. 1A-C depict the results of MLDA analysis of DNA found in a biological sample (colonic lavage) of human subjects examined for the presence of and stage of colorectal cancer. Each row represents one subject and each column throughout FIGS. 1A-C represents one allele. Coloration in each box denotes the frequency of individual alleles present in the sample. The number in each box denotes the single allele having the highest frequency in each sample (the dominant allele). FIG. 1A depicts MLDA analysis of subjects without detectable disease (n=24). FIG. 1B depicts MLDA analysis of subjects with adenoma (n=16) or with carcinoma in situ (n=6). FIG. 1C depicts MLDA analysis of subjects with colorectal carcinoma invasive. (n=21). (for actual values see Table S1).

FIGS. 2A-B depict the results of MLDA analysis of DNA found in a biological sample (solid stool) of human subjects examined for the presence of and stage of colorectal cancer. Each row represents one subject and each column throughout FIGS. 2A-B represents one allele. Coloration in each box denotes the frequency of individual alleles present in the sample. The number in each box denotes the single allele having the highest frequency in each sample (the dominant allele). FIG. 2A depicts MLDA analysis of subjects without detectable disease (n=4). FIG. 2B depicts MLDA analysis of subjects with colorectal carcinoma invasive. (n=4).

FIGS. 3A-C are graphs demonstrating the aggregate (or total) mutational load of human subjects analyzed using the MLDA methods described herein. The total mutational load parameter derived from the MLDA analyses demonstrates the ability to distinguish four groups of subjects (non-neoplastic disease, adenoma, carcinoma in situ and carcinoma invasive); there is a narrow band of overlap between adenoma and carcinoma in situ, and there is a narrow band of overlap between carcinoma in situ and carcinoma invasive. The increase in total mutational load can be seen as a reflection of progressive genetic instability. Samples obtained from colonic lavage during the colonoscopy are represented with filled circles (.); samples from colonic lavage prior to colonoscopy (cathartic samples) are represented with crosses (x) and the samples obtained from solid stool are represented with open circles (o). FIG. 3A depicts the total mutational load in each subject for all alleles examined. FIG. 3B depicts the total mutational load in each subject for all k-ras alleles examined. FIG. 3C depicts the total mutational load in each subject for all p53 alleles examined.

FIG. 4 demonstrates the correlation between samples obtained with colonic lavage and samples obtained from colorectal tissue by comparing the profile of the percentage altered alleles in a biological sample obtained from solid stool sample (labeled “stool” and indicated by short dashes) as compared to a biological sample obtained from a solid tumor sample (labeled “biopsy” and indicated by long dashes) of the same human subject. Each human subject is labeled Sample A, B or C. The 22 mutations k-ras and p53 analyzed are presented on the x-axis, and percentage value of each mutated allele is presented on the y-axis. Sample A indicates a 100% correlation between results obtained from tissue and stools. Samples B and C show that 2-3 alleles differ in signal intensity range in biological samples obtained from tissue and stools. This difference in signal intensity range indicates that DNA obtained from bowel lavage is predominantly derived from neoplasm-exfoliated cells, although the remaining large bowel mucosa also contributes to overall MLDA. (For actual values see Table S-4).

FIGS. 5A-B depict the results of MLDA analysis of DNA found in a biological sample (pancreatic juice) of human subjects examined for the presence of and stage of pancreatic cancer. In FIG. 5A, each row represents one subject; the upper panel is composed of subjects with no known pancreatic pathology, the middle panel groups patients with chronic pancreatitis at increased risk for pancreatic carcinoma and the lower panel depicts the results obtained in patients with pancreatic carcinoma. Each column throughout the panels represents one allele and the color in each box denotes the frequency of the corresponding allele making up the molecules encoding Ki-ras p21 or the p53 protein. Although many alleles in cancer patients were above 5% the actual representation is cut-off in order to depict the dynamic range of values between 0 and 5%. The MLDA profiles as well as the aggregate mutational load value clearly separate the three groups.

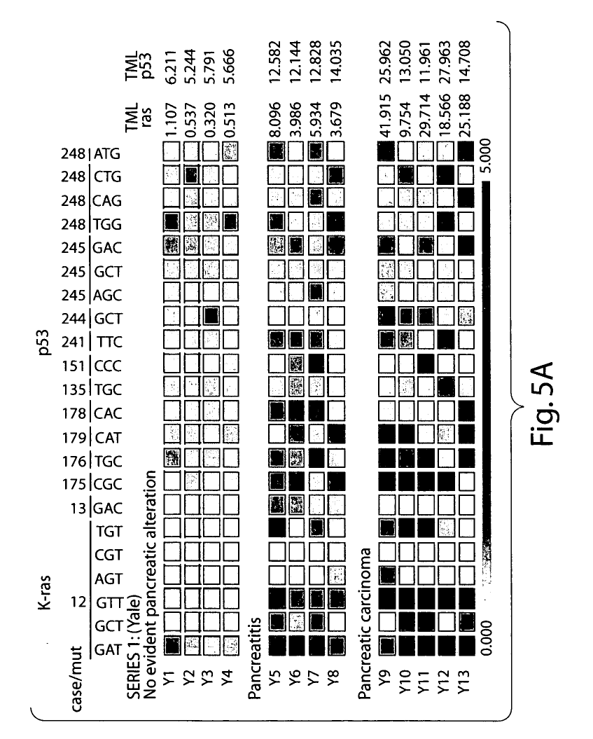

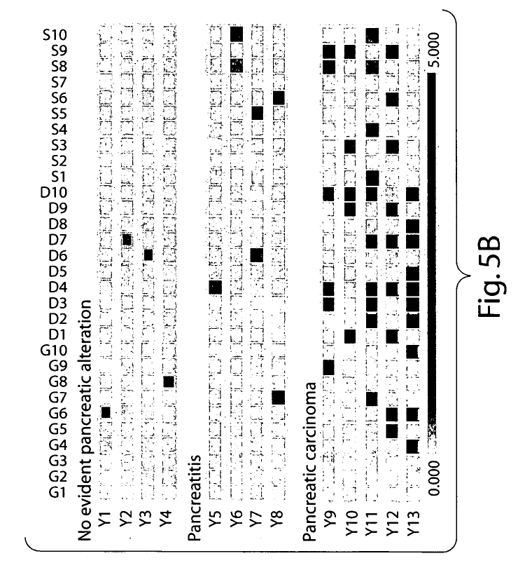

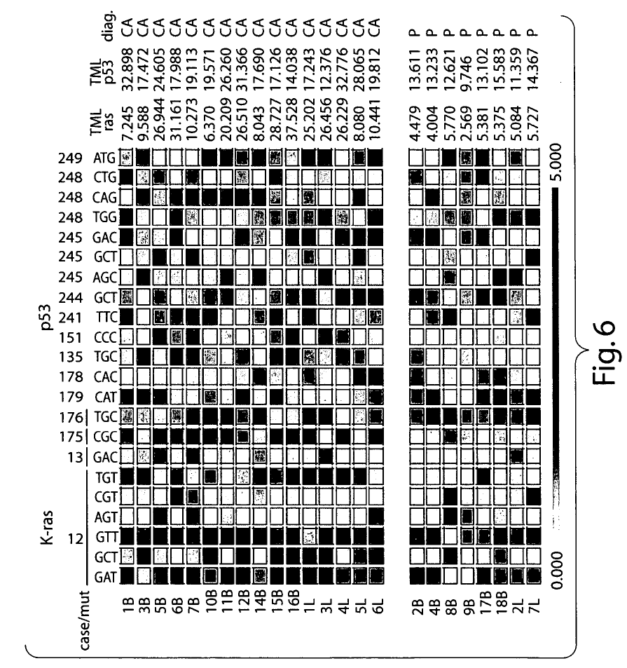

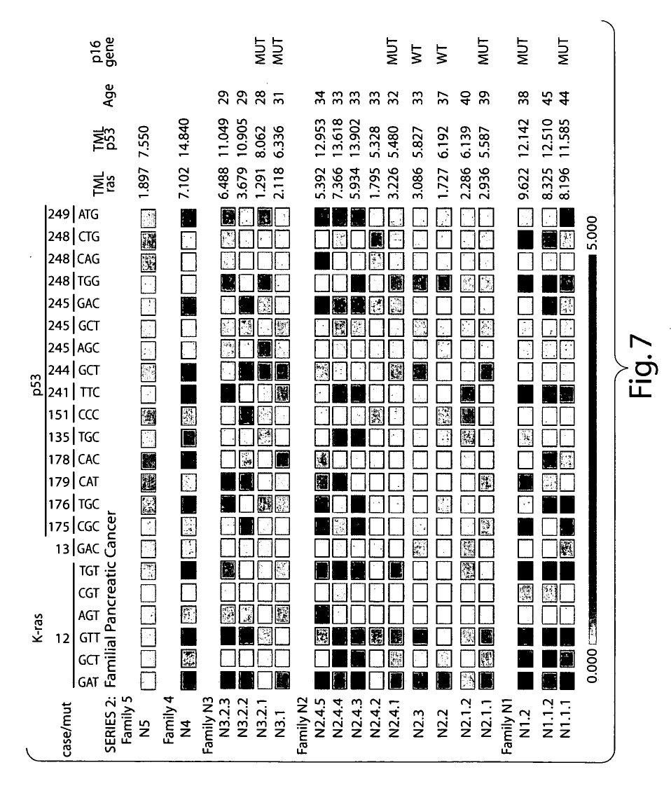

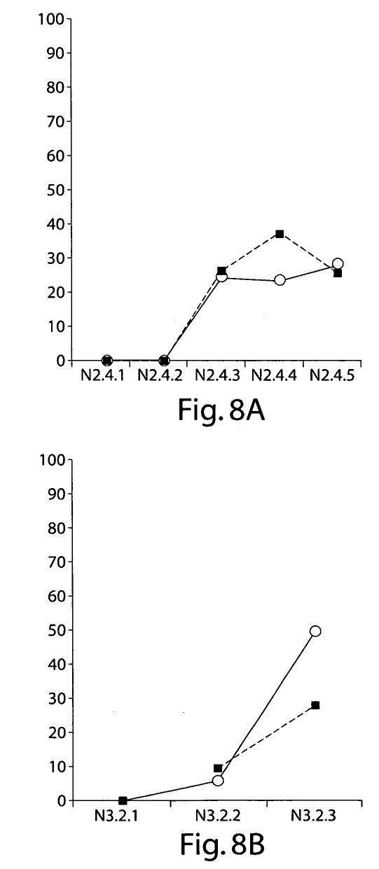

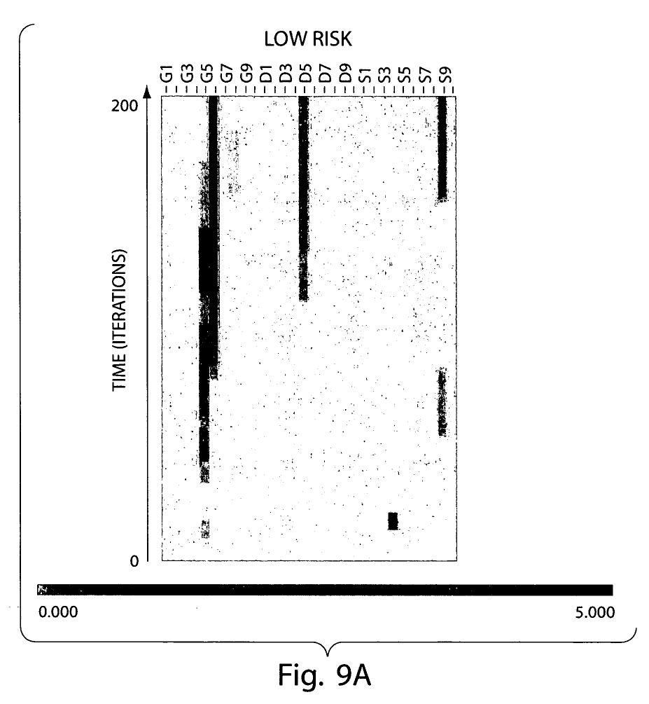

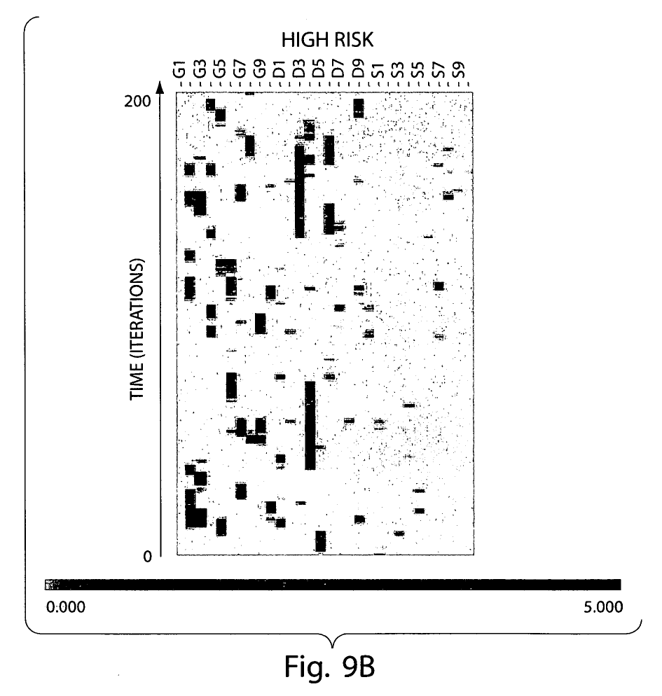

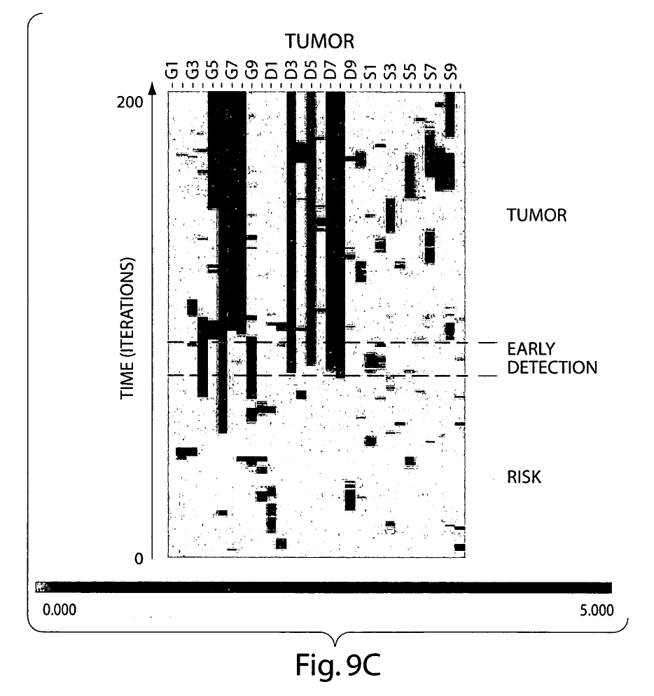

In FIG. 5B, columns represent the mutational load for 10 alleles of 3 genes (proliferative rate, first 10 columns; death rate, middle 10 columns, susceptibility to disturbance, last set of 10 columns). Each row represents a single run, showing 4 or 5 runs for each group. The mutation rate and the fitness parameters were identical for all groups. Only the disturbance frequency and intensity differs among the groups. The upper panel represents subjects having a low risk (no cancer); the middle panel represents subjects having a high risk (no tumor formation occurred for the duration of simulation); the lower panel represents subjects having a pancreatic tumor (tumor formation defined as accumulation of 3 mutations in an expanding clone at any time during simulation).

FIG. 6 is a chart that demonstrates the ability of MLDA profiles to distinguish pancreatic cancer from pancreatitis. Presentation of data is as described for FIG. 5A. The ability to classify cases using the MLDA metrics is shown in this case series suggesting that the threshold values defined by the first cohort are clinically valid.

FIG. 7 is a chart representing MLDA profiles from subjects belonging to different families with increased risk for pancreatic cancer due to inherited p16 mutation. Two patterns are recognized in the samples: a normal-like pattern (e.g., family 5, N5) and a pancreatitis-like pattern (e.g., family 4, N4). For subjects with sequential samples the profiles vary from normal like to pancreatitis like indicating an increase in risk. Note that in instances when the risk increases the alleles with high values do not necessarily persist. The total load for Ki-ras and p53, the age at the time of sampling and the p16 genotype are provided for each subject on the right.

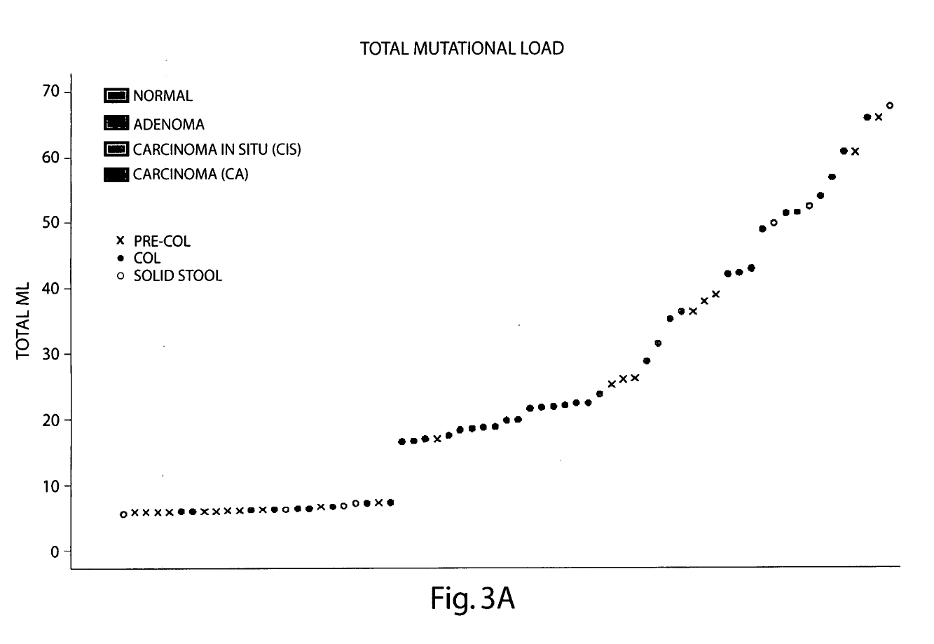

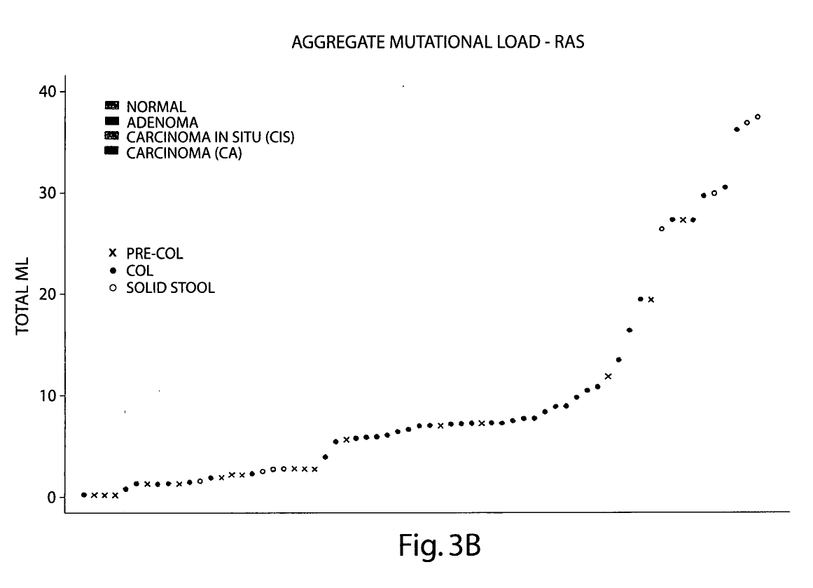

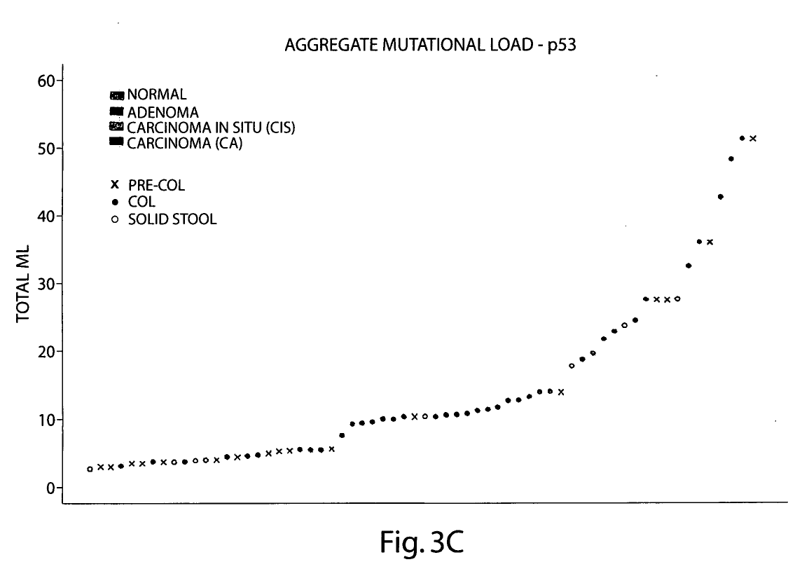

FIGS. 8A-B are line graphs showing variations in risk level with time for two of the subjects depicted in FIG. 3. The MLDA metrics can be translated to degrees of risk based on the boundaries defined by the initial studies. (Ki-ras mutations are represented by o—o; p53 mutations are represented by ▪-▪).

FIGS. 9A-C are simulated images depicting simulated mutational load over time. Graphic representation of the MLDA values obtained by play-back of values at each time step in runs for the three classes of outcome. Time series of mutational load at each 25th step over the entire 5000 iterations (200 time points per run). Rows represent the mutational load at a single time point proceeding from bottom (t=0) to top (t=5000). Columns represent the mutational load for 10 alleles of 3 genes as in FIG. 5. FIG. 9A represents a simulated mutational load of low risk subjects (no cancer, also termed “undisturbed”). FIG. 9B represents a simulated mutational load of high risk subjects (no tumor formation for duration of simulation). FIG. 9C represents a simulated mutational load of subjects with a pancreatic cancer.

### DETAILED DESCRIPTION OF THE INVENTION

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

The present methods of using mutational load distribution analysis (“MLDA”) for cancer diagnosis and recurrence monitoring offer several advantages over what had previously been known in the art. MLDA of DNA found in bodily fluids yields biometrics that enables early cancer diagnosis. MLDA provides increased sensitivity and specificity of cancer detection; these values each approach 100% using the methods of the present invention. Also, the present invention allows for the discrimination not only between cancer and non-cancer, but among stages of cancer and allows the discrimination of the risk of an individual to have or develop cancer of a given stage. Further, although the art discloses the use of various tissues or fluid samples to perform MLDA, disclosed herein is the high degree of specificity regardless of the sample type used. In a preferred embodiment, stool MLDA is a useful non-invasive marker of a distal and proximal colonic neoplasms.

Definitions

For convenience, certain terms used in the specification, examples, and appended claims are collected here. Unless otherwise defined, all technical and scientific terms used herein have the same meaning as commonly understood by one of ordinary skill in the art to which this invention pertains. However, to the extent that these definitions vary from meanings circulating within the art, the definitions below are to control.

As defined herein, the term “allele” refers to any one of a series of two or more different genes that occupy the same position (locus) on a chromosome.

The term “HPA” refers to highest prevalence allele and means allele present in the greatest amount in any given sample.

The term “mutated allele” refers to an allele that possesses one or more nucleotide changes (e.g., a point mutation) or a deletion or insertion of one or more nucleotides in its nucleic acid sequence. A mutated allele also includes alleles containing modified DNA, e.g., DNA methylation, thymidine dimerization.

The phrase “frequency of a mutated allele” refers to the relative numbers of a given allele that is mutated relative to the numbers of the given allele that are nonmutated (wild type).

The phrase “reference frequency” refers to the frequency of a given allele of a gene in a reference population of subjects. The subjects of this reference population may or may not have cancer.

The phrase “proportion of mutated alleles” refers to the number of alleles that are mutated alleles, relative to the number of nonmutated (wild type) alleles.

As used herein, a “test sample” includes any organic material obtained from a subject, from which one or more alleles can be determined.

The phrase “degree of diversity” refers to the type of mutational change displayed in a mutated allele. For example, a mutated allele may display three types of point mutations at a specific locus, relative to the wild type (wild type=T; point mutations are C, G, or A). A high degree of diversity would result from all three point mutations occurring at equal frequency (essentially randomly). A low degree of diversity would result if a specific point mutation becomes favored relative to the wild type.

The term “correlating” refers to describing the relationship between the proportion of mutated alleles and the degree of diversity of mutated alleles for a selected allele. Such correlation may be displayed graphically, or may be displayed in tabular format.

The phrase “sufficient time” refers to any time period required to assess the risk of cancer development with reasonable accuracy (generally on the scale of weeks to years).

“Subject” includes living organisms such as humans, monkeys, cows, sheep, horses, pigs, cattle, goats, dogs, cats, mice, rats, cultured cells therefrom, and transgenic species thereof. In a preferred embodiment, the subject is a human. A subject is synonymous with a “patient.” Administration of the compositions of the present invention to a subject to be treated can be carried out using known procedures, at dosages and for periods of time effective to treat the condition in the subject. An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered daily or the dose may be proportionally reduced as indicated by the exigencies of the therapeutic situation.

“Substantially pure” includes compounds, e.g., drugs, proteins or polypeptides that have been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state. Included within the meaning of the term “substantially pure” are compounds, such as proteins or polypeptides, which are homogeneously pure, for example, where at least 95% of the total protein (by volume, by wet or dry weight, or by mole percent or mole fraction) in a sample is the protein or polypeptide of interest.

“Administering” includes routes of administration which allow the compositions of the invention to perform their intended function, e.g., treating or preventing cardiac injury caused by hypoxia or ischemia. A variety of routes of administration are possible including, but not necessarily limited to parenteral (e.g., intravenous, intraarterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 16th Edition, Mack, Ed. (1980)).

“Effective amount” includes those amounts of the compound of the invention which allow it to perform its intended function, e.g., treating or preventing, partially or totally, cancer or another disease or disorder characterized by aberrant cell proliferation, as described herein. The effective amount will depend upon a number of factors, including biological activity, age, body weight, sex, general health, severity of the condition to be treated, as well as appropriate pharmacokinetic properties. For example, dosages of the active substance may be from about 0.01 mg/kg/day to about 500 mg/kg/day, advantageously from about 0.1 mg/kg/day to about 100 mg/kg/day. A therapeutically effective amount of the active substance can be administered by an appropriate route in a single dose or multiple doses. Further, the dosages of the active substance can be proportionally increased or decreased as indicated by the exigencies of the therapeutic or prophylactic situation.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

“Pharmaceutically acceptable esters” includes relatively non-toxic, esterified products of therapeutic compounds of the invention. These esters can be prepared in situ during the final isolation and purification of the therapeutic compounds or by separately reacting the purified therapeutic compound in its free acid form or hydroxyl with a suitable esterifying agent; either of which are methods known to those skilled in the art. Acids can be converted into esters according to methods well known to one of ordinary skill in the art, e.g., via treatment with an alcohol in the presence of a catalyst.

“Additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, e.g., in Remington's Pharmaceutical Sciences.

General Description of the Invention

Somatic mutations result from seemingly random environmental mutagenesis and are often followed by expansion of the allele within a clonal population of cells. The vast majority of such clones die before they accumulate additional mutations or before they expand further under the pressure of a selection mechanism. It is this fluctuation that is observed by the methods of the present invention as random drift in the frequency of mutated alleles. Thus, for a randomly mutated normal population, the mutational load distribution is broad. Conversely, with the emergence of a single clonal population of cells carrying a given allele (an oncodeme) that expands many fold against the same background population, a loss of mutational load diversity is observed. Therefore, by measuring altered (e.g., mutated or polymorphic) alleles in a tissue or organ, and determining any expansion of these alleles within a cell population over time, one is able to predict the location of where a tumor is likely to emerge. The determination of either the proportion or diversity of mutated cancer gene alleles, or both, in samples that represent a large population of cells from an organ or tissue using the methods disclosed herein, one is able to evaluate the acquired cancer risk for the subject as well as identify the stage and metastatic potential of the cancer of which the subject is at risk.

As used herein, a mutation includes any change in a nucleic acid (e.g., DNA or RNA) that can be reproduced. Generally, a mutation in a subject's genomic DNA will involve the change in sequence of one or more nucleotides. Mutations include point mutations (such as substitutions, transitions, and transversions), insertions, and deletions. Mutations involving multiple nucleotides include inversions and rearrangements. For use of MLDA in cancer diagnosis, any gene implicated in cancer by mutation can be assessed. Examples include point mutations leading to the gene either being inactivated or activated. Specific examples of genes to be assessed for colorectal cancer include apc, k-ras and, p53. In addition to MLDA analysis using point mutations, MLDA can also be used to assess DNA which has been modified post-synthetically. For example, DNA methylation is a common form of DNA post-synthetic modification in which a cytosine-guanine base pair is modified by the addition of a methyl group. DNA methylation is associated with regulation of expression of the methylated gene. Therefore DNA hypo- or hyper-methylation changes can be used with the method of the present invention to provide information for diagnosing cancer, staging cancer, or monitoring the recurrence of cancer.

Methods for Analyzing Cancer Stage and Progression

The present invention is based, in part, on the ability to differentiate cancer cells from normal (i.e., non-cancerous) cells by analyzing certain DNA mutations or polymorphisms.

In particular, it has been found that the proportion of mutated alleles in cancer cells from the subject, or the total mutational load of the cancer cells, are useful in predicting or determining the stage of the subject's cancer, or monitoring the recurrence of cancer. Thus, one aspect of the invention provides a method of evaluating the risk of cancer development in a subject that includes the following steps:
(1) providing from the subject a test sample of material for which the risk of cancer development is to be evaluated;
(2) quantitating the proportion of one or more mutated alleles in the test sample, relative to one or more nonmutated alleles; and
(3) comparing the proportion of the one or more mutated alleles in the test sample with a reference proportion.

As described herein, the inventor has discovered that when a higher proportion of one or more mutated alleles is observed in the test sample than in the reference proportion, the subject from whom the sample was provided either has cancer or has an elevated risk of cancer. The afore-mentioned steps are described in greater detail below.

The Total Mutational Load (TML) of a limited number of selected mutational hotspots, for example, in K-ras and p53 genes and the highest prevalence allele (HPA) provide information that is highly predictive of the state of the colonic epithelium including the identification of benign and malignant neoplastic lesions, irrespective of their location. In addition to mutations in k-ras and p53 genes, mutations in any cancer related genes can be used. As opposed to conventional biomarkers that are based on a single or multiple tumor-specific targets, MLDA exploits the sensitive and quantitative assessment of mutation to use the intrinsic variability within non-tumor and tumor tissues as a source of information. Multiple quantitative assessments are key in enabling the discrimination of different pathological states of progression (adenoma-CIS/invasive carcinoma).

This analysis of variability in mutant allele prevalence offers a balanced sampling of the entire length of the colon and allows the correct classification of all normal mucosae due to the low variance in TML and HPA metrics present in cells originating from endoscopically normal mucosa. By distributing the mutational load in 22 alleles belonging to two genetic loci, MLDA diminishes the false positive results associated with the detection of mutations in Ki-ras or p53 in the absence of pathological lesions (Imperiale TF, et al. N Engl J Med 2004; 351:2704-14).

Fecal MLDA has allowed the identification of all tumors, irrespective of their location in distal or proximal colon. Interestingly, two distinct MLDA profiles underlie a high TML value. When highly predominant allele/s are present MLDA probably reflects the result of strong selection acting on a low level of genetic instability. Alternatively, when a high TML metric results from uniformly high values equally distributed throughout the alleles examined it is likely to reflect a high level of genetic instability or the possibility that the probe for the dominant allele was not included in the panel. In the latter setting MLDA overcomes the intrinsic limitation of conventional markers that miss the tumors failing to harbor specific mutations suggesting that quantitative assessment of multiple alleles is a key factor. Even the use of a multiplicity of markers, scored as present or absent (Imperiale TF, et al.), would fail to encapsulate the information derived from quantitative variational metrics in MLDA.

For this purpose the use of robust, quantitative and sensitive analytical techniques that allow the definition of consistent quantitative thresholds is critical.

The majority of MLDA data reported here derive from colonic lavages, a source of readily amplifiable DNA. Results obtained in a limited set of samples extracted from solid stool suggest that MLDA could be more widely applicable provided that consistent and efficient DNA extraction techniques are used (Whitney D, et al. J Mol Diagn 2004; 6:386-395., Tarafa G, et al. Mutational load distribution yields metrics reflecting genetic instability and selection during pancreatic carcinogenesis. Submitted.). Finally, the comparison between tissue and fluid MLDA corroborates the notion that fecal DNA tests obtained with no diet modification can provide relevant information derived from the entire length of the colon (Osborn NK and Ahlquist DA. Gastroenterology 2005; 128:192-206).

In average-risk population, fecal DNA testing detected a greater proportion of relevant benign or malignant tumors than FOBT (Imperiale TF, et al.). Fecal DNA is becoming a practical alternative to FOBT. In our preliminary experience 4 of 4 normals and 2 of 3 carcinomas analyzed were FOBT positive. Altogether, our approach opens new vistas to the use fecal DNA as the analyte in the non-invasive diagnosis of colorectal carcinoma due an initially encouraging accuracy in discriminating between normal mucosa and any type of advanced neoplasia.

Accordingly, fecal MLDA can be a useful strategy to overcome the intrinsic limitations of single or multipanel strategies and thus contribute to the early detection of colorectal cancer, pancreatic cancer or any type of cancer. None of the fecal tests reported to date (Ahlquist DA, et al. Gastroenterology 2000; 119: 1219-27, Imperiale TF, et al., Sidransky D, et al. Science 1992;256:102-105, Puig P, et al. Int J Cancer 2000;85:73-77, Eguchi S, et al. Cancer 1996;77:1707-1710, Traverso G, et al. N Engl J Med 2002; 346: 311-20, Traverso G, et al. DNA. Lancet 2002; 359: 403-4) has yielded such initially encouraging results regarding sensitivity and specificity and correlation with corresponding biopsies.

Sample procurement and preparation

The present invention provides test samples from a subject. The subject can be a human, a non-human mammal, or any animal. Any test sample that contains cells from the subject or any cellular material that contains a nucleic acid from the subject is suitable for use in the present invention. Thus, any body tissue or body fluid may be used as a sample source of DNA for organs or anatomical regions where mutations are to be quantitated. In preferred embodiments, the test sample is a colonic lavage, a cathartic preparation, a stool sample, or a colonic brushing. In other preferred embodiments, the test sample is blood, a tumor biopsy, a tumor aspirate, a cultured tumor cell, or bone marrow. Examples of other useful tissues or fluids include sputum, pancreatic fluid, bile, lymph, plasma, urine, cerebrospinal fluid, seminal fluid, saliva, breast nipple aspirate, pus, biopsy tissue, fetal cells, amniotic fluid, and the like. Preferably, fluids derived from pancreas (ERCP aspirates), breast (nipple aspirates or nipple lavages), or colon (stool) are selected because of the possibility of obtaining surrogate fluids that contain cells and cellular material representative of the cell population (e.g., epithelial cells) from which cancer originates. Fluids can be collected from patients at risk for cancer using protocols and methods well known in the art.

For example, DNA can be isolated with relative ease from the fluid and cells obtained by endoscopic retrograde cannulation of the pancreatic duct. For breast, collecting nipple fluid yields cells and biological material from a wide basin. Active aspiration of the nipple yields approximately 50 microliters of fluid from which cells, protein and soluble DNA are obtained (Sauter E. R., Cancer Epidemiology, Biomarkers & Prevention 7:315-320, 1998), and which results in nanogram-range quantities of DNA. For colon, it is possible to perform cell brushings from small areas of mucosa during colonoscopy. Using this procedure, DNA samples from the interior of the colon may be obtained. DNA from colon is extracted directly from colon cells present in a stool sample. Tissue samples may be obtained by laser capture microdissection.

Generally, nucleic acids (e.g., DNA) are extracted from the test sample. Although the method of the present invention is preferably implemented with DNA as a source for mutations, alternative nucleic acids, such as RNAs, may also be used in the method of the present invention. Accordingly, the invention is not intended to be limited by the source of nucleic acids in the samples. DNA thus extracted is quantitated and stored in aliquots containing diploid genome equivalents. Cytological specimens from brushings or fluids are fixed in a fixative solution or on slides in a way that preserves the material for the identification of mutations. In embodiments of the invention, different fractionation procedures can be used to enrich the test samples for specific molecules such as nucleic acids. The molecules obtained are then be passed over one or several fractionation columns or other nucleic acid separation means. Following sample preparation, each of the samples is then analyzed for mutations including point mutations and/or microdeletions using the methods described below.

Allele detection In accordance with the method of the present invention, following test sample isolation and preparation, the proportion of mutated alleles and the degree of diversity of mutated alleles in the sample are quantitated. In one embodiment, the step of quantitating the proportion of mutated alleles is done by first identifying the mutated alleles, relative to wild type (normal) alleles using techniques described below, and scoring (e.g., counting) the number of alleles with mutations. Similarly, in one embodiment, the step of quantitating the degree of diversity of mutated alleles in the sample may be performed by identifying the type of mutation relative to the wild type, and scoring that mutation. In general, the steps directed to quantitating the proportion of mutated alleles and the degree of diversity of mutated alleles in the sample may be performed by any method known in the art; preferably, the method is a sensitive, quantitative, and efficient (i.e., high throughput) procedure that can simultaneously assess mutations in many alleles in cell populations the size of an oncodeme. Preferably, the selected method or methods will be capable of (1) detecting specific point mutations, microdeletions, or hyper- or hyper-methylations in a quantitative fashion; (2) testing a large number of samples; and (3) have a sensitivity at the level of detection of 1% of altered alleles in a background of wild type alleles. Examples of useful technologies for mutational analysis in accordance with the method of the invention include rolling circle amplification techniques, beacon array techniques, and comparative genomic hybridization. Each of these methods are described in more detail below. Any gene that, when mutated, is associated with the onset and/or progression of cancer (termed “cancer-associated” genes) can be analyzed using the methods of the present invention. These genes include oncogenes, proto-oncogenes, and tumor suppressor genes, and family members of such genes. In embodiments of the invention MLDA is performed using multiple alleles of a given cancer-associated gene. The present inventor has identified a number of genes containing mutated alleles that are informative in determining the proportion of mutated alleles and the mutational load, including K-ras, p53, APC, and BAT26. By way of non-limiting example, the invention discloses several mutated alleles in Table 1. However, other genes containing mutated alleles can be used in the methods of the invention by those skilled in the art.

TABLE 1
mutated alleles

| Gene name | codon name | Type of mutation |
|---|---|---|
| K-ras | codon 12 of K-ras | GAT |
| | | GCT |
| | | GTT |
| | | AGT |
| | | CGT |
| | | TGT |
| | codon 13 of K-ras | GAC |
| | | TAC |
| p53 | codon 135 | CGC |
| | codon 151 | TGC |
| | codon 175 | CAT |
| | codon 176 | CAC |
| | codon 178 | TGC |
| | codon 179 | CCC |
| | codon 241 | TTC |
| | codon 244 | GCT |
| | codon 245 | AGC |
| | | GCT |
| | | GAC |
| | codon 248 | TGG |
| | | CAG |
| | | CTG |
| | codon 249 | ATG |

Disclosed in Table 1 by way of non-limiting example are 23 alleles of two cancer-associated genes. The present invention provides for methods that examine any number of alleles of any number of genes, e.g., cancer-associated genes. For example, MLDA is performed using 1, 2, 5, 8, 10, 15, 18, 19, 20, 21, 22, 23, 24, 25, 26, 28, 30, 35, 40, 50 or more alleles.

MLDA can also be performed by analysis of DNA methylation markers to predict the presence, recurrence, or stage of cancer in a subject. (See example 4 for additional details.).

Methods of Allele Detection

Rolling Circle Amplification (RCA)

In one embodiment, rolling circle amplification (RCA) techniques may be used to quantitate the proportion and degree of diversity of mutated alleles as described in Ladner et al., Laboratory Investigation 81:1079-1086 (August, 2001). Briefly, rolling circle amplification driven by a strand-displacing DNA polymerase can replicate circularized oligonucleotide probes with either linear or geometric kinetics under isothermal conditions (Lizardi, P. M. et al., Nature Genetics, 19:225-232,1998). Using a single primer, RCA generates hundreds of tandemly linked copies of the circle in a few minutes. If matrix-associated, such as in arrays or cytological specimens, the DNA product remains bound at the site of synthesis where it may be fluorescently tagged, condensed and imaged as a point light source. Hybridization of a target sequence to immobilized and arrayed oligonucleotides can be visualized as single hybridization events and quantitated by direct molecular counting. When allele discriminating oligonucleotides are used to catalyze specific target-directed ligation events, wild type and mutant alleles can be discriminated as each allele generates a different fluorescent color signal when amplified by RCA. Thus, when used in an array format, RCA is particularly amenable for the analysis of rare somatic mutations and the study of mutational load.

In RCA, oligonucleotide probes are hybridized to complementary DNA targets and circularized by ligation. This ligation reaction may be exploited. for allele discrimination, or may be used to copy part of the target sequence into the circularized DNA. Using a single primer, complementary to the arbitrary portion of the circular DNA, a strand-displacing DNA polymerase (from phage .PHI.29) may be used to generate DNA molecules containing hundreds of tandemly linked copies of the covalently closed circle. In general, it takes less than 20 minutes to generate several hundred copies of the circular DNA template. When rolling circle DNA replication is carried out in the presence of two suitably chosen primers, one hybridizing to the (−) strand, the other to the (+) strand of the DNA, a geometrically expanding cascade of sequential DNA strand displacement reactions ensued, generating 10⁹ or more of copies of each circle in 90 minutes. This geometrically expanding cascade is called Hyperbranched Rolling Circle Amplification (HRCA). HRCA can be used to detect, among other things, point mutations at a specific locus of the CFTR gene in small amounts of human genomic DNA (Lizardi, P. M. et al., supra). Like PCR, the Hyperbranched RCA reaction is capable of generating hundreds of millions of copies of a single DNA probe molecule. Therefore, HRCA is primarily useful for solution-based genetic analysis. For detection applications on the surface of microarrays, the linear, single primer reaction is a more attractive approach.

In one embodiment, RCA is useful for generation of individual “unimolecular” signals that may be localized at their site of synthesis on a solid surface. The DNA generated by a rolling circle amplification (RCA) reaction can be detected on a surface as an extended single strand, or as a condensed, tightly coiled “ball”. Cross linking reagents and fluorescence labeling may be used to permit observation of small spherical fluorescent objects of tightly condensed DNA arising from the amplification of a single circularized oligonucleotide (Lizardi, P. M. et al., supra). The individual signals are approximately 2 to 0.7 microns in diameter, and are easily imaged using an epifluorescence microscope with a tooled CCD camera.

There are two alternative approaches for the use of localizable RCA signals in gene detection. The first approach consists of using a circularizable probe (called the Open Circle Probe) to interrogate the target sequence of interest (Lizardi, P. M. et al., supra). The second approach consists of using a pre-existing circular DNA of arbitrary sequence, to extend a primer that is bound to a target on a surface of the primer is linked covalently to a detection probe, which defines target recognition specificity, while the circle is merely a reagent for a subsequent amplification reaction. Generally, the probe-primer may contain any probe sequence. The circular DNA oligonucleotides, as well as the primers, contain arbitrary sequences. Because in this system the primer is a generic reporter that can be amplified by RCA, it is also possible to implement assays where the detection “probe” is an antibody capable of binding a specific antigen. As mentioned above, RCA can be used for the generation of individual “unimolecular” signals that may be localized at their site of synthesis on a solid surface. Simple procedures known in the art using cross linking and fluorescence labeling permit observation of small spherical fluorescent objects that consist of a single molecule of amplified DNA. In this embodiment, multiple analytes may be detected using either DNA sample arrays, or oligonucleotide arrays. These types of applications require optimized surface chemistry, multicolor labeling protocols and DNA condensation methods, which are described below.

A strategy for detection of DNA targets using derivatized glass surfaces has been described and is known in the art (Lizardi, P. M. et al., supra). Briefly, the method exploits the capability for localizing RCA signals originating from single DNA primer molecules. Genomic DNA mixed in different ratios Is amplified by PCR and hybridized on slides with immobilized probes, in the presence of an equimolar mixture of two allele-specific probes in solution. After a hybridization/ligation step, ligated probe-primers are detected by RCA. The images show many hundreds of fluorescent dots with a diameter of 0.2 to 0.6 microns, which are generated by single condensed DNA molecules. The ratio of fluorescein-labeled to Cy3-labeled dots corresponded remarkably closely to the known ratio of mutant to wild type strands, down to a value of 1/100. The Single Molecule Counting method is based on target-dependent ligation of reporter allele-specific probe-primers on a glass slide surface.

Fluorescence in situ Hybridization (FISH)

In situ methods may also be used to detect mutations in alleles. In one embodiment, DNA fibers may be used in conjunction with fluorescence in situ hybridization (FISH) techniques to detect mutations in alleles. Briefly, DNA fibers are prepared from cultured fibroblasts or lymphoblasts from normal individuals and individuals with homozygous or heterozygous mutations at the G542X locus of the cystic fibrosis gene using conventional DNA stretching techniques (Heiskanen M, et al., Genomics 30:31-36 (1995)). 1000-5000 cells in PBS buffer were spotted onto the end of a clean microscope slide, and the cells lysed for 5 minutes by the addition of an equal volume of 0.2% SDS. The slide was placed in a Coplin jar in a vertical position and the cell lysate allowed to dribble down the surface by gravity and then air dried. The sample was then fixed in methanol-acetic acid (3:1) for 10 minutes, washed, air dried and then treated with 0.1 mg/ml proteinase for 30 minutes, rewashed and air dried.

Molecular Beacons

Molecular beacons are structured DNA probes that generate fluorescence only when hybridized to a perfectly complementary DNA target. The utility of these probes for the detection of specific sequences in PCR amplicons has been widely documented (Tyagi, S. et al., Nature Biotechnology 14:303-308 (1996); Tyagi, S., et al., Nature Biotechnology 16:49-53 (1998)). Molecular beacons may be immobilized on solid surfaces, where they function with the same excellent sequence specificity (Ortiz, E., et al., Molecular and Cellular Probes, 12:219-226 (1998)). Notably, immobilized beacons offer much larger potential for multiplexing relative to beacons used in solution. An important feature of molecular beacons is their improved capacity for allele discrimination, as compared to linear probes. The beacon stem provides an alternative stable structure that competes successfully with a mismatched hybrid, and thus the beacons remain in the quenched (closed) conformation even in the presence of target DNA capable of forming a mismatched hybrid. Allele discrimination ratios of 70:1 have been documented for many loci (Marras S. A. et al., Genet. Anal. 14:151-6 (1999); Bonnet, G. et al., Proc. Natl. Acad. Sci. USA (1999)). Molecular beacon arrays also offer advantages in terms of cost, reusability, and simplicity.

Immobilized molecular beacons are generally derived from oligonucleotides synthesized with a 3′-terminal DABCYL moiety, a reactive aminolinker side chain, a stem of 5 bases, a probe domain of 18 to 20 bases and a stem-complement of 5 bases, terminating with a fluorescent residue at the 5′-end. Some of the original molecular beacons utilized fluorescein as the fluorophore. However, dyes which are less susceptible to photobleaching are generally preferred. Most notable among these are the ALEXA dyes (Molecular Probes, Inc.) which combine high fluorescence yield with high resistance to photobleaching. The oligonucleotide synthesis generally takes place in an automated synthesizer using standard phosphoramidite chemistry using standard reagents. Oligonucleotides are aliquoted on standard microtiter dishes at a concentration of about 200 □M. They are then dispensed as small droplets on the surface of activated glass slides (20 nanoliters per droplet) using the microarraying robot. Standard glass microscope slides are pre-activated with monomethoxysilane, generating a derivatized monolayer harboring the functional group 1,4-phenyler adiisothiocyanate. The primary amine in the second position of the molecular beacon oligonucleotide reacts with the derivatized glass surface, generating arrays with a high coupling efficiency (1×10¹¹ beacon molecules per square mm).

Comparative Genomic Hybridization (CGH)

Comparative genomic hybridization (CGH) has become a powerful tool for assessing chromosomal abnormalities (genetic losses and gains) in a broad spectrum of tumors. CGH has been used to determine genetic alterations in a variety of tumor types and at various stages of progression. However, the major limitation of CGH is the level of resolution obtained using metaphase chromosomes as the endpoint readout. Recently, it has been demonstrated (Pinkel, D., et al. Nature Genetics. 20:207-11 (1988)) that cohybridization of reference and sample DNAs to an array of cloned (and mapped) genomic DNA can provide higher resolution analysis of copy number variation in tumor specimens. In using such clone arrays and the inclusion of sufficient control parameters for hybridization efficiency and specificity, differences in fluorescent ratios of clones represented in the tumor DNA at one, two or three copies per cell could be detected.

The performance criteria for array CGH (A-CGH) are more stringent than those of related array-based methods for measuring levels of gene expression. Single copy gene changes relative to the normal diploid state must be detected as reliably as large copy number changes. Since the entire genome is used as a hybridization probe, it is between 10 to 20 fold more complex than those used to profile expressed sequences and it contains significant amounts of highly repetitive sequence elements. Pinkel, et al. (supra) added various amounts of 1 DNA to reference human genomic DNA to define the sensitivity and quantitative capability of their A-CGH protocol. Using cosmid, P1, BAC and other large insert clones as array targets, Pinkel, et al. demonstrated that the measured fluorescence ratios were quantitatively proportional to copy number over a dynamic range of 200-500 fold, beginning at less than 1 copy per cell equivalent. The hybridization of two different samples of genomic DNA (one tumor and one normal), each labeled with a different fluorophore, to an array of cDNA clones in order to establish their relative DNA copy number has recently been reported (Pollack, J. et al., Symposium on DNA Technologies in Human Disease Detection, San Diego, November 1998). These investigators were able to demonstrate an analytical sensitivity sufficient to detect a two-fold change in DNA copy number, equivalent to the detection of low level DNA amplification or allele loss. Significantly, this approach provides the opportunity to monitor gene expression and DNA copy number changes in the same sample.

The method of the present invention implements a similar strategy using either cDNA clones or, preferably, synthetic oligonucleotides, to form an array of genes or ESTs from the chromosomal regions described above. The number of mapped cDNAs and EST markers has increased dramatically over the past few years thus making it feasible to synthesize defined oligonucleotide probes spanning large segments of the genome. A unique feature of the method of the present invention is the use of rolling circle amplification (RCA) technique in an immunodetection mode to markedly increase the sensitivity of hybrid detection. Genomic DNA from the tumor cells, e.g., a small set of cells constituting a potential oncodeme, can be labeled by nick translation or random priming with biotinylated nucleotides. Control reference cell DNA can be labeled similarly using digoxigenin nucleotides. Post-hybridization detection can be done using “immuno-RCA”, a method recently shown to be capable of visualizing single antigen-antibody complexes in a manner analogous to the detection of single DNA-oligonucleotide hybridization events. Antibiotin antibody can be covalently coupled to an oligonucleotide that will form the primer for RCA amplification of a preformed circle. Antibodies to digoxigenin can be labeled with a different oligonucleotide sequence that will prime RCA on a second circle sequence. The resultant RCA products, reflecting amplification from the hybridization of tumor DNA (biotin) or control (Digoxigenin) DNA, can be distinguished by using two RCA detector probes labeled with different fluors. Two color ratio imaging of RCA products should define the relative copy number of genes within the sample. Using immuno-RCA to visualize and count individual oligonucleotide-genomic DNA hybridization events should both enhance the sensitivity of detection of A-CGH and provide a higher resolution analysis than large clone arrays. As gene map densities increase, immuno-RCA should permit copy number ratio imaging on a gene by gene basis.

Oligonucleotide probes are generally selected by sequence analysis of chromosomal regions known to display loss of heterozygosity (LOH) or gene amplification in cancer lesions. Candidate sequences will be compared to Genbank entries using the BLAST program, in order to find sequence domains that represent unique, single copy sequences with no known homologues at other chromosomal loci. Only unique sequences will be selected for inclusion in the arrays. The length of the sequences will be 60 bases to permit very stringent washing after array hybridization.

Data Correlation

Following quantitation of the proportion of mutated alleles and the degree of diversity of mutated alleles, the data is correlated to determine the risk of cancer development. This is done by comparing the proportion of the one or more mutated alleles in said test sample with a reference proportion. Generally, the reference proportion is a proportion derived from data generated by performing MLDA on a population of one or more subjects that are known to not have cancer or an elevated risk of cancer.

As indicated above, correlating means establishing a relationship between the proportion of mutated alleles and the degree of diversity of mutated alleles for a selected allele. In the method of the present invention, a preferred type of relationship is one in which, for a specific allele, there is an increase in the proportion of this particular allele, relative to the wild type, and a concomitant decrease in the diversity of mutations at that allele. In other words, a natural selection occurs such that a particular mutation becomes dominant and is preferred for a particular allele. Simultaneously, there may be a decrease in the mutational load of one or more other alleles, such that the total mutational load remains the same as a randomly mutated population.

The quantitating and correlating steps of the method of the present invention are repeated over a period of time and the particular locus is monitored for proportion of mutated alleles and degree of diversity. Preferably, the steps of the method of the present invention are repeated 2 to 10 times, and at intervals ranging from 6 times per year (every other month) once every two years, and more preferably twice per year to once per year. As indicated above, it is difficult to determine whether a particular mutated allele will mature into a malignancy by simply identifying the mutation because the background of normal mutational occurances and complexity significantly masks those true premalignant clones that are likely to progress into cancer. By repeating the steps of the method of the present invention over time, a pattern of identifiable alleles will emerge that are likely to progress into cancer. The data collected on each evaluation can be stored and compared over time to evaluate the risk of cancer. It is worthwhile to note that even genes with no direct relevance to cancer are useful in this analysis, since to a first approximation somatic mutational events target all genes randomly. Thus the method of the present invention can focus on genes of known tumor relevance, and additional applications of this method can achieve ever increasing levels of sensitivity and discrimination by analyzing larger gene panels.

Colorectal Cancer

While it is recognized that the methods described herein are generally applicable to all cancers, the present inventor has determined that these methods are particularly beneficial in evaluating the risk of colorectal cancer development in a subject and determining the stage of colorectal cancer in the subject. As demonstrated in Example 1, the methods of the invention allow the discrimination of multiple types of colorectal cancer, including adenoma, carcinoma in situ and invasive carcinoma. A preferred test sample contains an exfoliated cell, such as an epithelial cell that has sloughed off from the colorectal cancer. Further, non-invasive methods of obtaining test samples are disclosed. As described in Example 1, results of MLDA performed using a stool sample is about as reliable as when the test sample is obtained from a colonic brushing, a more invasive method of sample procurement.

Pancreatic Cancer

The present inventor has also determined that these methods are particularly beneficial in evaluating the risk of pancreatic cancer development in a subject and determining the stage of pancreatic cancer. As demonstrated in Example 2, the methods of the invention allow the discrimination of multiple types of pancreatic cancer, including pre-cancerous pancreatitis and pancreatic carcinoma. Moreover, the methods of the present invention allow for the identification of subjects at risk for pancreatic cancer due to familial susceptibility. A preferred test sample contains pancreatic juice obtained by canulation of the pancreatic duct. Alternatively, the test sample is a bodily fluid obtained after stimulation of the subject with secretin.

Cancer Recurrence Analysis

The present invention provides methods for the early detection of a cancer recurrence in a subject following treatment of the subject for the cancer. For example, a subject is treated by surgically removing all or essentially all of a solid tumor. At one or more times following this removal, a tissue sample is taken from the subject and analyzed with MLDA. A subject without cancer recurrence has a frequency of one or more alleles of a gene below a given reference frequency. However, a subject whose cancer has recurred will have an increased frequency of one or more mutated genes relative to a reference frequency.

Candidate Drug Screening

The present invention provides for the identification of subjects (e.g., humans) at risk for developing a given stage of a cancer. This allows for the generation of a population of subjects to test candidate anti-cancer drugs. By identifying those subjects likely to be affected by an anti-cancer drug, screening efficiency is increased over the methods currently known in the art.

Populations of Nucleic Acids and Kits

The invention provides populations of nucleic acid molecules that contain mutated alleles in genes associated with cancer. In embodiments of the invention, the population of nucleic acid molecules contains a first nucleic acid molecule and a second nucleic acid molecule, wherein the first and second nucleic acid molecules each contain a mutated allele obtained from a cancer-associated gene such as K-ras, p53, APC, or BAT26. These populations are useful, e.g., to obtain clinical information of the status of a tumor or cancer in a subject, such as the likelihood that the tumor or cancer will progress to a more malignant stage. In some embodiments, the populations are used when the subject is a human suffering from or is at risk of cancer. In embodiments, the nucleic acid molecules are covalently bound to a solid or semi-solid support medium, such as an array. In other embodiments, the population further comprises a means for detecting one or more mutated alleles

The invention also provides kits containing a population of nucleic acid molecules containing a first nucleic acid molecule and a second nucleic acid molecule, means for obtaining from a subject a test sample, and instructions for use thereof. In embodiments of the invention, the first and second nucleic acid molecules of the kit each contain a mutated allele obtained from a cancer-associated gene such as K-ras, p53, APC, or BAT26. The means for obtaining the test sample includes any means capable of collecting blood, urine, a tumor biopsy, a tumor aspirate, a cultured tumor cell, bone marrow, a stool sample, or a colonic brushing. In some embodiments, the kit also include a means for calculating the proportion of mutated alleles in a sample from the subject, or the total mutational load of the sample.

### EXAMPLES

### Example 1: MLDA Analysis of Colorectal Cancer in Human Subjects

The methods of the present invention were performed on a population of human subjects (termed “patients” herein) suffering from or at risk of developing a colorectal cancer.

Patients Accrual and Stool Collection

A total of 67 samples from two centers (Institut Catalá d'Oncologia and Hospital de Sant Pau) were included. Forty (9 normal, 31 tumors) bowel lavage fluid samples were collected during performance of screening colonoscopies after positive FOBT testing. The remaining 20 (11 normal, 9 tumors) fluids were obtained immediately prior to colonoscopy from symptomatic patients after cathartic preparation. Finally, a set of 7 solid stools (4 normal, 3 tumors) of symptomatic patients undergoing a colonoscopy was collected. Final diagnosis was: 24 non-neoplastic diseases (6 inflammatory bowel disease, 9 colonic diverticulosis and 9 normal colonoscopies), 16 adenomas, 6 carcinomas in situ and 21 invasive carcinomas. All fluid samples were collected and immediately frozen and stored at −80° C. Biopsies of endoscopically evident lesions for which ravages were available were collected in 25 of the 37 tumors and an aliquot was frozen for MLDA studies. In one case with tumor, biopsies of three areas of endoscopically normal mucosa were also obtained. In four cases with no evidence of disease biopsies obtained from three distinct normal areas were available for analysis. A written informed consent was obtained from patients for their willingness to participate in this laboratory-based study, and the work was carried out after approval of the institutional reviews board at both participating centers.

Mutational Load and Distribution Analysis (MLDA).

DNA was extracted from cellular material obtained after centrifugation of bowel lavage or solid stools as previously described (Puig P, et al., Int J Cancer 2000;85:73-77, Puig P, et al. Lab Invest 79:617-618,1999.). An oligonucleotide zip-code micro-array with rolling circle amplification signal enhancement that enables the simultaneous quantitative interrogation of tissue fluids for a moderate number of alleles was used (Ladner DP, et al. Lab Invest 2001; 81: 1079-86.). Alleles of both the Ki-ras and p53 genes are well suited for stool MLDA since both are altered in a significant proportion of colorectal neoplasms (Olivier M, et al. Hum Mutat 2002; 19: 607-14.). We selected 22 mutations, 7 in exon 1 of the K-ras gene and 15 in exons 5 and 7 of the p53 gene that were both prevalent enough and technically compatible for being interrogated simultaneously (Ladner DP, et al.).

Fiftying of genomic DNA were used to PCR-amplify Ki-ras exon 1 and p53 exons and 7 in a final volume of 30 microliters. Amplified DNA was used for a multiplex ligation detection reaction (LDR). LDR products were hybridized onto generic zip code 3D-Link slide microarray and detected by rolling circle amplification decorated with complementary fluor-oligonucleotides. Slides were scanned at 635 nm on a GSI Lumonics 4000 Scanarray and analyzed with Spot (CSIRO, Mathematical Information Analyses, Australia). The array was composed of 12 subarrays each containing 3 replicates of any interrogated mutation as well as 3 replicates of printing controls and three reconstituted controls with serial dilutions of a known mutation that allowed for quantitation of the total number of mutant alleles (Ladner DP, et al.). Normalization of a given sub-array was performed using the signal intensity of three sample-control replicates and the added intensity of all controls. Trimmed median values of the intensities of the 36 replicates for a given mutation were used to make all calculations. The intensities of all alleles interrogated for a given nucleotide were added and percentages were obtained. The distribution of the alleles was represented in a color scale grading. In order to assess reproducibility MLDA hybridizations of a pool of 3 colorectal carcinomas (TML=41,53) were independently repeated. Mean SD was 0.055. When duplicates were performed in 14 samples (4 normal, 5 adenomas and 5 carcinomas) mean SD of MLDA was 0.048 confirming the robustness of the assay. Since, after adjusting for diagnosis, no significant differences were observed between results obtained from fecal DNA of colonic lavages obtained prior to or during colonoscopy or solid stools, a joint analysis of all samples was performed. All laboratory results were read without knowledge of clinical status.

Statistical Analysis

To assess the predictive accuracy of TML as a metric derived from MLDA in distinguishing the different groups, two approaches were used. A training set of the first 40 samples analyzed (9 normal, 15 adenomas and 16 carcinomas) was used to define the halfway cut-off point between the maximum values of normal and the minimum of neoplasia. This value was used in a testing set including the remaining samples (15 normal, 1 adenoma and 11 carcinomas; 20 lavage and 7 solid stools) to confirm sensitivity and specificity.

Secondly, a modeling statistical approach using all data was used to confirm the predictive accuracy of TML. A logistic regression model was chosen to build a predictive rule for the diagnosis. When building discriminant models to differentiate normal from adenomas and from carcinomas, polytomous logistic regression was used. In order to explore whether a subset of mutations could account for most of MLDA information, stepwise logistic regression and random forest analyses (Breiman, L. Machine Learning 2001; 45: 5-32.) were used. To properly estimate the misclassification error rates, accounting for overfitting, 10-fold cross-validation and bootstrap techniques were used (Efron B and Tibshirani R. J Am Stat Assoc 1997; 92: 548-560). Throughout the manuscript we have followed the STARD recommendations for reporting studies of diagnostic accuracy (Bossuyt P M, et al. Clinical Chemistry 2003; 49:7-18).

Results

The arrays we utilize enable precise quantitation of the alleles present in a DNA sample expressed as the allele prevalence (calculated as %). The profile of the percentage of all the abnormal alleles constitutes a mutational load distribution for a given sample and yields two biometrics: total mutational load (TML)-calculated after adding the prevalence of mutant alleles for every mutation—and highest prevalence allele (HPA). The combined analysis of these variables is termed “Mutational Load Distribution Analysis” (MLDA).

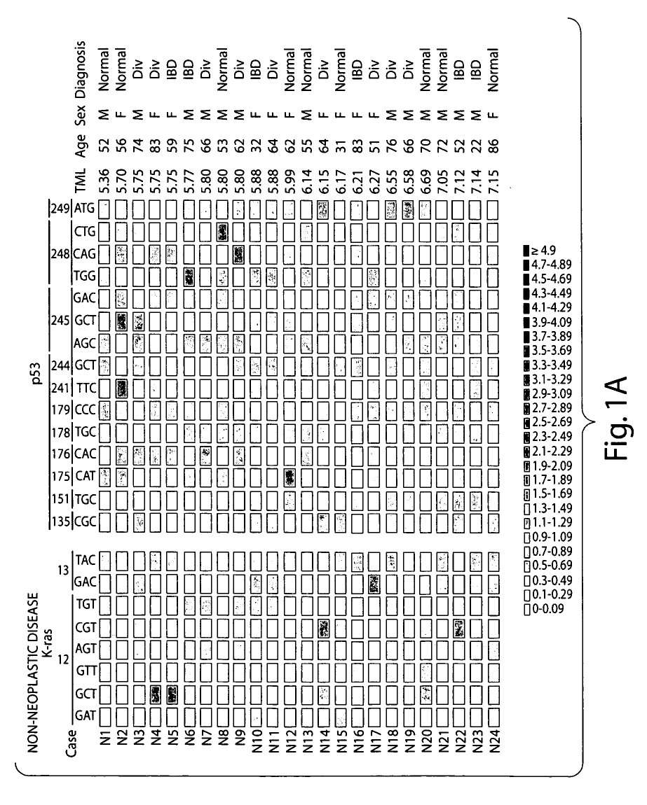

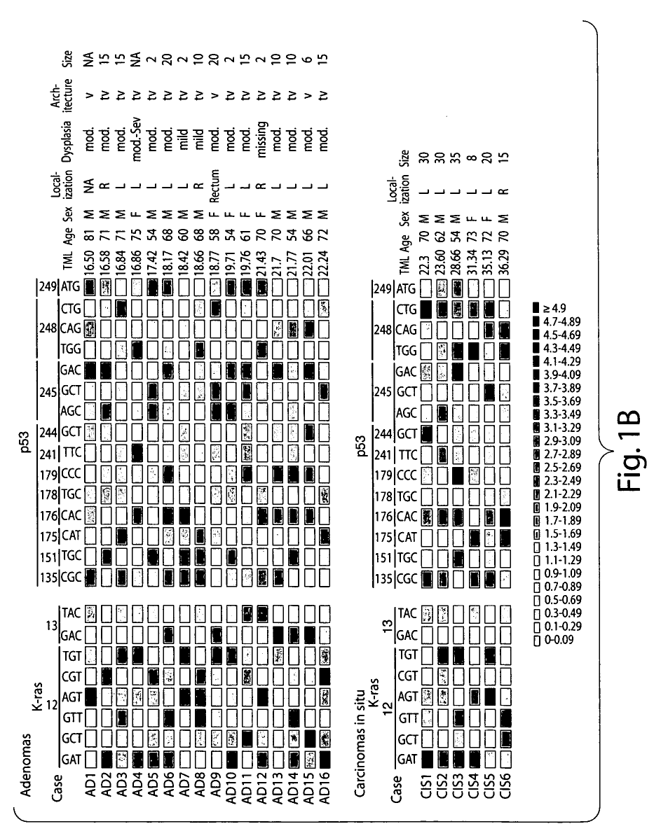

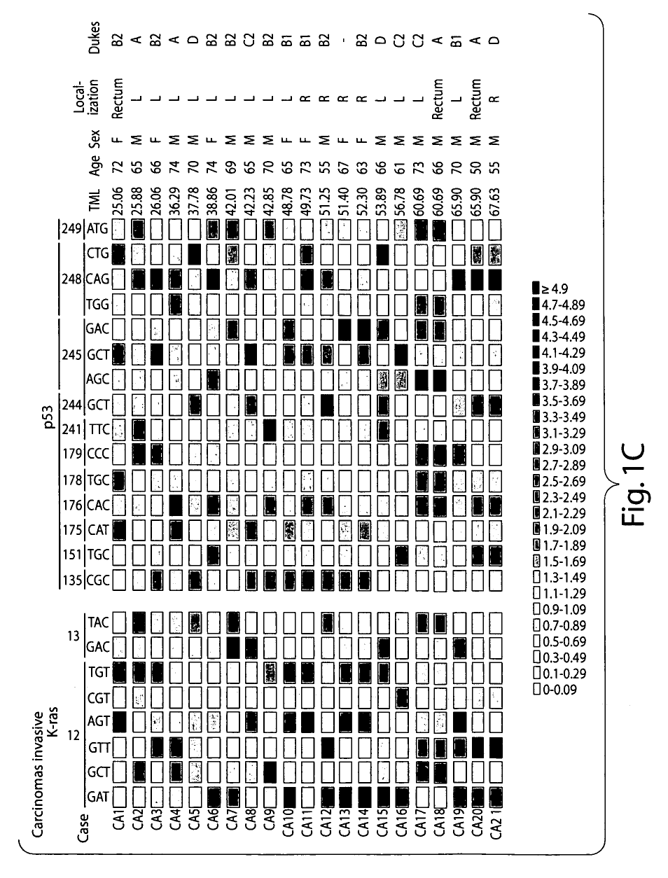

The MLDA profiles obtained for all the cases studied are shown in FIG. 1 (Table S1). Inspection of the patterns suggests that the different categories of individual examined can be easily distinguished. TML of non-neoplastic disease ranged from 5.3 to 7.15 (average 6.18) and no single mutant allele constituted more than 1.2% of the population of molecules examined for a given nucleotide. TML of adenomas ranged from 16.50 to 22.24 (average 19.17) with several alleles (range 5-9 out of 22) showing prevalence higher than 1.2% but never exceeding 9.5%. TML of carcinomas in situ ranged from 22.30 to 36.29 (average 29.5) and TML of invasive carcinomas ranged from 25.06 to 67.9 (average 47.71). Single mutant alleles showing prevalence above 12.3% associated with carcinoma although in some cases (3 of 21) they were not present. TML clearly discriminated non-neoplastic disease from tumors and a progressive increase in TML can be observed through the adenoma-carcinoma sequence (FIG. 3). Adenomas and carcinoma in situ clustered together with a trend towards increased load in the latter and invasive carcinomas appear as a distinct category (FIGS. 1 and 3).

HPA identified two categories: (i) carcinoma defined by an HPA of 12.3% or higher (malignant dominant allele); and (ii) adenoma or carcinoma in situ characterized by an HPA representing 1.2 to 9.5% of the molecules (“benign dominant” allele). SSCP analyses confirmed the presence of a Ki-ras dominant allele mutation in 11 of 13 cases analyzed. The two cases with HPA lower than 6% could not be confirmed (data not shown). In four cases with a p53 dominant allele, mutation was confirmed by direct sequencing.

To preliminarily assess the predictive accuracy of MLDA the population was split into two sets. In the training set the halfway TML cutoff value for the presence of any neoplasm—either benign or malignant—was 11.87. Using this cutoff value sensitivity and specificity was 100%. When applied to the independent set, sensitivity and specificity were again 100% respectively. Using the complete set of samples, and taking into account the potential overfitting, the estimated sensitivity was 100% (95% Cl 91.7-100) (46/46) and specificity was 100% (95% Cl 86.2-100) (24/24).

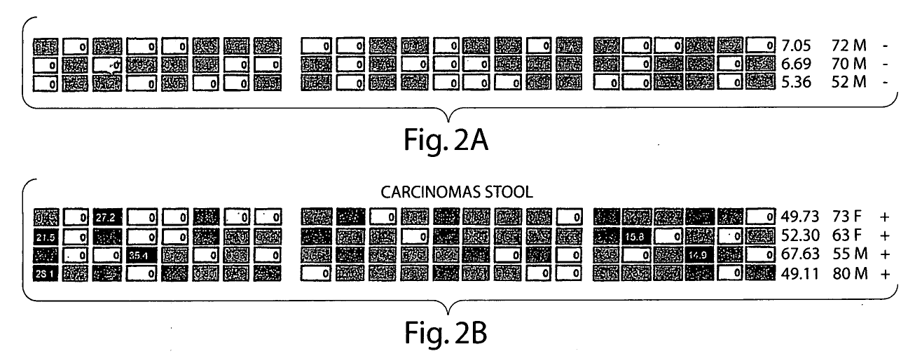

Although MLDA-derived metrics in carcinomas were always greater than in adenomas, there was imperfect separation between benign and invasive lesions (FIG. 2). The misclassification error rate estimated from bootstrap re-sampling was 2%, corresponding to an average of 1 misclassified individual: almost systematically, an individual with in situ carcinoma was classified as adenoma.

No specific subset of mutations included in the panel of probes used for MLDA could account for MLDA information derived from the entire set. Though stepwise logistic regression identified a set of mutations that perfectly discriminated normal from pathologic samples, the misclassification error rate after bootstrapping was 30% when three categories (normal, adenoma and carcinoma) were considered. Accordingly, random forest analysis of all data created a final tree with a 30% misclassification error rate. Interestingly the relative importance of all mutations in the construction of the tree was similar suggesting that no specific mutation was especially informative.

Correlation Between Stools and Tissues

In each of 4 cases with no evidence of disease we analyzed three biopsies (ascending, traverse and descending colon) that were confirmed to have normal architecture by histology. For each case, all three samples yielded metrics that belong to “no-disease” category (variation coefficients ranging 5-35%) (Table S2). When DNA from the three biopsies was pooled, MLDA metrics strongly resembled that of the corresponding solid stool (mean TML difference between pairs 0.16 representing 6% of the average TML value found in the stool samples) suggesting that fecal MLDA offers a balanced representation of the colonic epithelium.

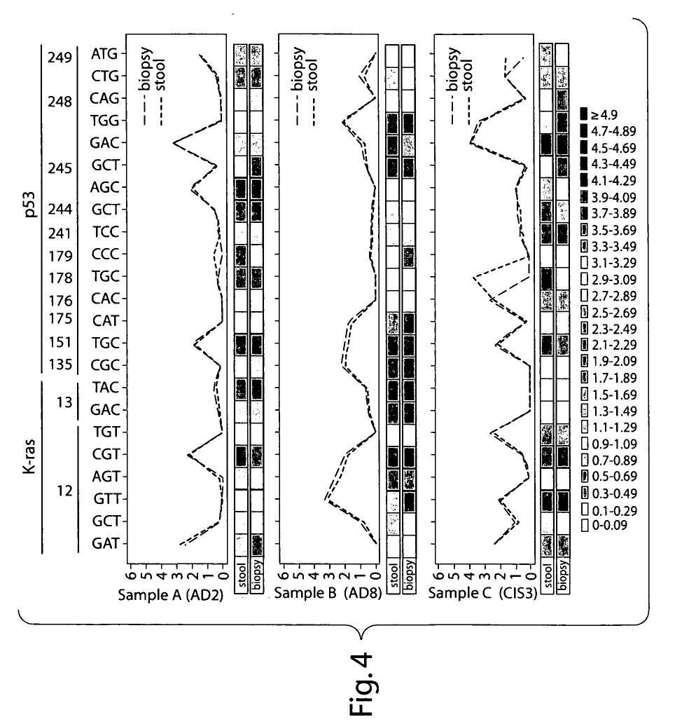

MLDA profile of tumor biopsies and corresponding stool samples showed a high degree of correlation (Pearson r=0.992) (FIG. 4; Table S4). Biopsies of normal mucosa in a tumor-bearing patient showed the profile and metrics of the normal class. (Table S2). In 11 of 14 adenomas and in 8 of 11 carcinomas an average of 2-3 alleles, out of the 22, gave discordant prevalence values. Interestingly in 4 adenomas and 2 carcinomas novel HPA of the “benign dominant” class appeared in stools (FIG. 3; Table S4). Thus, information contained in fecal DNA mainly derives from neoplasm-exfoliated cells the remaining large bowel mucosa also contributing to MLDA metrics.

Correlation Between Stools and Tissues.

MLDA was performed to compare biological samples obtained from biopsies and stool samples. Biopsies from 15 subjects having adenomas and 10 subjects having carcinomas were provided. An extremely high correlation was obtained between tissue and bowel lavage samples regarding total mutational aggregate and its distribution (FIGS. 2 and 4). TML was slightly higher in tissues usually associated with higher values of the dominant alleles. Also, allele distribution was slightly different in stools when compared with biopsies. In 13 of 15 adenomas and in 7 of 10 carcinomas an average of 2-3 alleles gave distinct signal intensity range. Interestingly in 4 adenomas and 2 carcinomas novel benign dominant alleles appeared in stools. This observation suggests that information contained in DNA from bowel lavage mainly derives from neoplasm-exfoliated cells although the remaining large bowel mucosa also contributes to overall. Finally in one case (AD7) that harbored two adenomas, both lesions and 3 normal biopsies of the descending, transverse and right colon were analyzed. TML of both lesions (20.12% and 19.85%) was similar to fecal MLDA (18.17%). In contrast average MLDA of normal biopsies was 6.09%. (See Table S2).

Feasibility in Solid Stools.

To further explore the feasibility of our approach a small set of selected solid stools (4 colorectal carcinomas and 4 normal endoscopy) that also has had FOBT (FIG. 2) were studied. Again MLDA correctly discriminated between carcinomas and lack of disease. Interestingly in two cases MLDA correctly identified a normal mucosa whereas FOBT yielded a positive result.

The analyses presented in this example demonstrate that MLDA clearly discriminates between normal mucosa and neoplastic growth—either benign or malignant—due to the low degree of dispersion in the total and distribution values of mutations present in cells originating from otherwise endoscopically normal mucosa. Interestingly a sequential increase in the total ML becomes apparent during the adenoma-carcinoma sequence with a further increase in invasive carcinomas. This trend shows some overlapping between adenoma and carcinoma in situ. However, no clinical impact for type of misclassification can be envisioned. While both ras MLDA and p53 MLDA independently contribute to the differential diagnosis a clear distinction between normal and neoplastic disease is evident when combining data obtained from both genes. It is intriguing that MLDA of carcinomas shows a high degree of variability, a finding that leaves open the possibility of MLDA values, probably reflecting the degree of genetic instability present in the tumor, may relate to clinical aggressiveness.

As opposed to conventional biomarkers that are based on a single or multiple targets that are specific for the tumor cell, MLDA exploits the quantitative assessment of mutation to use the intrinsic variability—heterogeneity—within tumor and non-tumor tissues as a source of information. The analysis of variability has allowed the correct classification—based on the total mutational load—of tumors that did not harbor a malignant dominant allele. Whereas conventional markers will miss the tumors falling to express the specific molecule(s), MLDA will report the emergence of any dominant tumor genotype. However the limited sample size analyzed may have introduced some bias (i.e. excess of K-ras positive and p53 mutations tumors) that should add caution to the interpretation of our results.

The use of robust, quantitative and sensitive analytical techniques in mutation detection has been also instrumental in this achievement. The reduced intra—and interassay variability and the low variance observed permits the definition of reliable quantitative thresholds that correctly discriminates between normal and neoplastic disease. Eventual technical developments in allelic discrimination are likely to help in reducing the number of replicates while improving throughput.

These results confirm previous observations suggesting that most of the information obtained by MLDA of stools come from tumor cells. Differences observed between stools and tumor biopsies probably reflect the contribution of exfoliated cells originating in other areas of the colon that have died and harbor mutations. It can be foreseen that the MLDA information contained derived from the normal epithelium will be helpful in evaluating the genetic stability of otherwise endoscopically normal mucosa prior to or after tumor development.

Feasibility of this type of assays in non-invasive samples is mandatory to change medical practice. The body of evidence reported derives from colonic lavages, a readily amplifiable sample difficult to obtain since it requires, at best, cathartic preparation. Our results in a limited set of solid stools show that MLDA strongly support its usefulness in the easy-to-obtain solid stools suggesting that this technique could be widely applicable provided efficient DNA extraction techniques are used. It is of note that in our hands, amplifiable DNA can be extracted in up to 80% of the samples using standard DNA extraction methods. As already noted for other fecal DNA tests, a single stool sample obtained with no diet modification can provide relevant information derived from the entire length of the colon.

Fecal DNA testing is expected to be a feasible alternative to conventional CRC screening strategies. So far, a multi-target panel is the best option available still hampered by a limited sensitivity for advanced adenomas and a modest decrease in specificity (16). Our approach seems to initially overcome most of these limitations.

### Example 2

### MLDA analysis of Pancreatic Cancer in Human Subjects

The methods of the present invention were performed on a population of human subjects (also termed “patients” herein) suffering from or at risk of developing a pancreatic cancer.

Patient Accrual and Stool Collection

Data in human subjects suffering from or at risk of developing pancreatic cancer was obtained by analyzing the soluble DNA found in pancreatic juice obtained by canulation of the pancreatic duct, or after stimulation with secretin.

An oligonucleotide zip-code micro-array with rolling circle amplification signal enhancement enables the simultaneous interrogation of tissue fluids for a moderate number of alleles (Bhatia et al. J of Clin One 2003: Vol 21, No 23; 4386-4394) and the detection of low prevalence allelic variants. Alleles of both the Ki-ras and p53 genes are well suited for MLDA of pancreatic juice (Olivier et al. Hum Mutat 2002 June; 19(6): 607-14; Hruban et al. Clin Cancer Res 2000a; Vol 6: 269-2972) since both are often found to be altered in a high proportion of pancreatic carcinomas. From the mutational spectrum of these two genes we selected 22 somatic point mutations (See FIG. 6) that were both prevalent enough to be informative and technically compatible for being simultaneously interrogated in an RCA enhanced zip-array format. Based on the known prevalence of the dominant alleles found in fully evolved malignant pancreatic tumors we predicted that we should be able to identify the emergence of 85% of cancers harboring a dominant Ki-ras clone and 70% of the tumors with a dominant p53 clone.

The ability of MLDA to discriminate among three distinct cohorts was determined. These cohorts included subjects without known pancreatic pathology or risk factors for pancreatic cancer, patients thought at increased risk for pancreatic cancer because of repeated bouts of pancreatitis, and patients with symptomatic pancreatic carcinoma. MLDA separated the three groups based on the aggregate value of the mutational load and on the level of the highest allele (See FIG. 5A). Among the subjects with no known pancreatic pathology, no single allele constituted more than 1.2% of the population of molecules examined. An allele constituting more than 3.8% indicated the presence of carcinoma and for all the cases of pancreatitis, category at risk, the frequency of the predominant mutant allele was found in the interval between 1.2 and 3.8%. Two dimensional plots of the aggregate and individual gene mutational load and multivariate linear estimates of the profiles obtained for the 22 alleles examined indicate that the differences observed are significant (differences among the three groups for Ki-ras p=0.004733; for p53 p=0.01458 Kruskal-Wallis test).

A comparison between the in silico simulation (See Example 3) and empirical data derived from patients with pancreatic cancer was performed and allowed the definition of boundaries that indicated a transition from normal to risk and risk to cancer. A cross sectional sampling of the different simulated populations, a low risk (undisturbed), a high-risk group (fraction of the disturbed population that does not develop tumors) and the fraction that develops tumors determines thresholds that separate the three groups by both the highest proportion of a mutated allele and the “aggregate mutational load”. As seen in FIG. 5B the empirical data and the data obtained from the simulation exhibit a strikingly similar pattern.

To test the clinical validity of the empirical cut-off values chosen based on the initial set of cases we blindly examined a retrospectively assembled set of samples comprising eight additional cases of pancreatitis and sixteen cases of pancreatic carcinoma. Seven of the 8 pancreatitis patients were identified as belonging to the risk group by both a distribution profile that revealed at least one allele above the 1.2% level but none above 3.8% and the aggregate mutational load. One case could be classified as “at risk” by the aggregate mutational load. Similarly all the pancreatic cancer patients were identified by the same two parameters with no false positive or false negative events, as shown in FIG. 6. When these groups are added to the initial ones the differences among the three categories remain significant (at the Ki-ras alleles, p=0.000001324 and for p53 alleles, p=0.0001162 using the Kruskal-Wallis test). The definition of the boundaries for the at risk for pancreatic cancer category indicates that it is possible to divide the interval between 1.2% and 3.8% in 100 equivalent segments to generate an arbitrary risk scale that should enable the longitudinal estimate of risk with the passage of time. To test this possibility we analyzed pancreatic juice from members of families predisposed to pancreatic cancer by a germ line p16 mutation. Blinded examination of the MLDA patterns in 16 samples showed two homogeneous groups: a “normal like” pattern and a “pancreatitis-like” pattern (see FIG. 7 legend) After un-blinding the series of samples and ordering them according to the individual of provenance, 4 individuals, harboring a p16 germ line mutation and belonging to 3 independent families, turned out to have iterative studies that provided data on the time dependent variation of MLDA derived parameters. The random fluctuation of the values for specific alleles obtained at different times can be appreciated in the serial samples of individuals exhibiting a normal like pattern as well as in some of the alleles in pancreatitis-like patterns. As can be seen in FIG. 7, of the six individuals with a p16 germ-line mutation, two had initial low risk samples and moved to the high-risk category, two were classified as “high risk” and remained in this class and two had a single time point study. It is important to note that the alleles that show the highest values vary from time point to time point. However in two instances the ascending allele remains identical suggesting an additional predictive factor for the development of cancer (See FIG. 7). FIG. 8 shows the risk estimates for two human subjects with serial samples. These observations underscore the value of using a wide mutational spectrum for each locus interrogated by MLDA. Not only is it impossible to predict which of the alleles will be driven by selection to be ultimately and predominantly expressed in the invasive tumor state but the allele that is dominant within the risk boundaries may vary due to chance events A disturbance, or a deleterious mutation, can eliminate an expanding oncodeme(s) and thus alter the subsequent MLDA pattern (see below and FIG. 9). Two individuals with normal p16 genotype showed profiles in the normal “no risk” zone.

In the absence of longitudinal empirical data that show the transition from high risk to tumor in a single subject, the in silico simulations enable us to validate the value of MLDA to serve as a biometric for the early detection of pancreatic cancer. For any specific run we can ascertain the in silico MLDA profile at each of the time steps for the entire time length of the simulation. Since the model is non-deterministic we can select runs that terminate in tumor formation and compare the MLDA profiles for each step to those of runs that terminate in tumor formation. We find that the MLDA profile does cross the “cancer threshold with no return” in the instances in which disturbance acts as a factor causing the emergence of a tumor (FIG. 9). Thus the results of the in silico simulation provide evidence of the measure of risk by longitudinal MLDA determinations. In the absence of empirical data that may take years to obtain, the model provides a strong argument to justify large prospective clinical validation studies for the measure of risk and early detection of tumors. The capacity of MLDA to provide a personalized longitudinal measure of risk, opens new vistas for the early detection of cancer and the monitoring of chemo-prevention.

Fluids derived from a subject is a useful test sample to obtain when practicing the methods contained herein. Generally, bodily fulids contain soluble DNA, and thus provide the means to repeatedly sample and monitor events occurring in the tissues without physical disruption. Because cells harboring mutations are more likely to die, either spontaneously or under the effect of disease (disturbance), the frequency of mutations found in fluids is higher than that expected in tissues. The results disclosed herein demonstrate that the aggregate mutational load, the proportion of the predominant mutated allele(s) and the persistence of dominance through time are informative parameters that are readily derived from MLDA analysis of pancreatic juice. As opposed to conventional bio-markers that are based on a single molecule (protein or nucleic acid) that is specific for the tumor cell, MLDA exploits variability as the source of information. Whereas conventional markers will miss the tumors failing to express the specific molecule, MLDA will report the emergence of any dominant tumor genotype. Most useful for longitudinal studies is the generation of a scale that enables the measurement of risk. The risk scale is based on the identification of two boundaries separating normal individuals from individuals at risk and the latter from patients harboring a tumor. Although not known at this point, we hypothesize that the values defining the boundaries depend in part on the size of the physiological clonal patches that form an adult tissue. For each organ (tumor type) to be studied by MLDA it will be necessary to determine the boundaries separating each category by conducting cross-sectional studies.

Risk measurement using MLDA in tissue or fluids from any material derived from a subject is applicable to any tissue or organ at risk of cancer. Breast cancer (nipple aspirates or ductal lavage), epithelial malignancies of the lower urinary tract (urine), broncho-pulmonary cancer (BAL) and others are potentially detectable at an early stage by MLDA.

### Example 3

### In silico MLDA Analysis

Herein described is an in silico simulation disclosing a stochastic model that explains the dynamics and distribution of mutational load and provides insight into the relation of parameters reflecting metapopulation dynamics to the emergence of tumors and therefore to the measure of cancer risk. The model, based on a micro-evolutionary view of carcinogenesis, takes into account intermittent global disturbances applied to a spatially structured tissue containing metapopulations of cells. Without disturbance, and for an arbitrary length of time representing the life span of the organism-host it is possible to parametrize the model in such a way that despite the occurrence of mutations no tumors emerge. Within a broad range of parameters we observed that intermediate frequencies and intensities of disturbance would lead to higher probabilities of tumor formation than in states with more extreme or no disturbances but with equivalent mutation rates, mutated phenotypes and otherwise identical model parameters. In the model, demes evolve on a grid with periodic boundary conditions. The fitness of a deme is a function of mutations affecting three general biological functions: the proliferative rate; the death rate (either promoting deme survival or more commonly by several orders of magnitude, deleterious to deme survival); and susceptibility to disturbances. Demes were initially randomly distributed throughout the grid at various densities. The parameters of a single run included a baseline mutation rate, wild type and mutated growth, death, and susceptibility probabilities, as well as disturbance frequency and intensity. Runs consisted of 5000 Monte Carlo iterations.

The simulations show that the hypothetical transition, from a randomly varying mutational spectrum to a spectrum persistently dominated by a pre-eminent allele(s), does take place during in silico carcinogenesis and distinguishes a population at risk from a population developing a tumor. Note particularly the similarity of the risk and tumor profiles during the early time period preceding the “early detection band”. The simulation indicates that the progressive increase in risk identifies the individual runs marked by the emergence of a “tumor”.

More importantly, “play back” of MLDA values for individual runs that result in tumor formation, shows that longitudinal MLDA can detect early stages of tumor development if applied in a prospective mode.

### Example 4

### DNA Methylation Analysis

The present invention also provides for the analysis of DNA methylation markers to predict the presence of cancer in a subject and the stage of cancer of the subject. Cytosine methylation occurs after DNA synthesis by enzymatic transfer of a methyl group from the methyl donor S-adenosylmethionine to the carbon-5 position of cytosine. About 70% of CpG dinucleotides in mammals are methylated during normal physiology; this amount and the specific CpG dinucleotides that are methylated changes over the development of cancer. The present invention provides for the measurement of DNA methylation in a subject suspected of having cancer or a predisposition thereto.

Methods of detecting DNA methylation include anti-mC antibodies, LC-mass spectroscopy, HPLC-TLC, Southern blotting, PCR, and the MethylLight assay. (See Eads et al., Nucleic Acids Research 28:e32 (2000); and Laird, Nature Reviews-Cancer 3:253-66 (2003).

DNA methylation markers include CDKN2A (ARF, INK4A); MLH1, APC, CDH1, CDKN2B, DAPK1, GSTP1, and MGMT. (See Laird, p. 261).

The preceding examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

TABLE S1

| gene | ras | ras | ras | ras | ras | ras | ras | ras | p53 | p53 | p53 | p53 |
|---|---|---|---|---|---|---|---|---|---|---|---|---|
| codon | 12 | 12 | 12 | 12 | 12 | 12 | 13 | 13 | 135 | 151 | 175 | 176 |
| mutation | GAT | GCT | GTT | AGT | CGT | TGT | GAC | TAC | CGC | TGC | CAT | CAC |
| N1 | 0.00 | 0.69 | 0.00 | 0.12 | 0.36 | 0.00 | 0.00 | 0.33 | 0.79 | 0.26 | 0.00 | 0.00 |
| N2 | 0.00 | 0.00 | 0.00 | 0.00 | 0.00 | 0.00 | 0.00 | 0.00 | 0.49 | 0.00 | 0.00 | 0.00 |
| N3 | 0.00 | 1.19 | 0.00 | 0.00 | 0.00 | 0.00 | 0.00 | 0.98 | 0.65 | 0.00 | 0.00 | 0.00 |
| N4 | 0.00 | 0.33 | 0.00 | 0.42 | 0.41 | 0.00 | 0.74 | 0.00 | 0.00 | 0.00 | 0.80 | 0.00 |
| N5 | 0.00 | 1.19 | 0.00 | 0.00 | 0.00 | 0.00 | 0.00 | 0.98 | 0.65 | 0.00 | 0.00 | 0.00 |
| N6 | 0.22 | 0.19 | 0.08 | 0.45 | 0.00 | 0.67 | 0.12 | 0.11 | 0.13 | 0.78 | 0.00 | 0.00 |
| N7 | 0.00 | 0.00 | 0.00 | 0.00 | 0.00 | 0.00 | 0.00 | 0.00 | 0.81 | 0.33 | 0.28 | 0.16 |
| N8 | 0.00 | 0.00 | 0.00 | 0.49 | 0.00 | 0.60 | 0.00 | 0.00 | 0.00 | 0.40 | 0.12 | 0.00 |
| N9 | 0.00 | 0.00 | 0.00 | 0.43 | 0.00 | 0.67 | 0.00 | 0.00 | 0.00 | 0.34 | 0.16 | 0.00 |
| N10 | 0.42 | 0.30 | 0.00 | 0.38 | 0.00 | 0.86 | 0.47 | 0.29 | 0.00 | 0.38 | 0.39 | 0.11 |
| N11 | 0.48 | 0.22 | 0.00 | 0.31 | 0.00 | 0.96 | 0.62 | 0.31 | 0.00 | 0.28 | 0.32 | 0.09 |
| N12 | 0.00 | 0.00 | 0.00 | 0.00 | 0.00 | 0.00 | 0.00 | 0.00 | 0.00 | 1.09 | 0.00 | 0.81 |
| N13 | 0.00 | 0.00 | 0.00 | 0.00 | 0.00 | 0.00 | 0.00 | 0.00 | 0.00 | 0.88 | 1.10 | 0.00 |
| N14 | 0.00 | 0.76 | 0.00 | 0.97 | 0.11 | 0.00 | 0.28 | 0.22 | 0.33 | 0.56 | 0.65 | 0.00 |
| N15 | 0.73 | 0.00 | 0.00 | 0.26 | 0.92 | 0.00 | 0.00 | 0.88 | 0.23 | 0.29 | 0.77 | 0.00 |
| N16 | 0.00 | 0.00 | 0.21 | 0.00 | 0.00 | 0.40 | 0.00 | 0.53 | 0.90 | 0.00 | 0.30 | 0.91 |
| N17 | 0.00 | 0.00 | 0.00 | 0.00 | 0.00 | 0.00 | 1.17 | 0.18 | 0.74 | 0.00 | 0.00 | 0.00 |
| N18 | 0.00 | 0.00 | 0.25 | 0.00 | 0.00 | 0.36 | 0.00 | 0.58 | 0.99 | 0.00 | 0.35 | 0.88 |
| N19 | 0.33 | 0.00 | 0.25 | 0.00 | 0.00 | 0.00 | 0.00 | 0.00 | 0.99 | 0.23 | 0.21 | 0.75 |
| N20 | 0.00 | 0.79 | 0.93 | 0.00 | 0.38 | 0.46 | 0.00 | 0.00 | 0.76 | 0.00 | 0.00 | 0.00 |
| N21 | 0.55 | 0.00 | 0.00 | 0.43 | 0.00 | 0.33 | 0.92 | 0.64 | 0.22 | 0.48 | 0.00 | 0.56 |
| N22 | 0.00 | 0.00 | 0.00 | 0.00 | 1.18 | 0.00 | 0.00 | 0.00 | 0.77 | 0.31 | 0.89 | 0.67 |
| N23 | 0.43 | 0.41 | 0.00 | 0.28 | 0.00 | 0.37 | 0.91 | 0.50 | 0.16 | 1.05 | 0.12 | 0.78 |
| N24 | 0.95 | 0.45 | 0.15 | 0.86 | 0.00 | 0.16 | 0.79 | 0.72 | 0.36 | 0.00 | 0.66 | 0.00 |
| AD1 | 0.00 | 0.00 | 0.15 | 3.39 | 0.00 | 0.70 | 0.00 | 1.32 | 0.00 | 0.31 | 2.19 | 0.00 |
| AD2 | 2.81 | 0.23 | 0.00 | 0.25 | 2.12 | 0.00 | 0.15 | 0.41 | 0.33 | 0.56 | 0.09 | 1.91 |
| AD3 | 0.55 | 0.34 | 1.65 | 0.91 | 0.00 | 2.07 | 0.25 | 0.88 | 0.93 | 0.71 | 1.5 | 0.00 |
| AD4 | 1.95 | 0.00 | 0.15 | 0.86 | 0.00 | 2.16 | 0.00 | 0.72 | 0.36 | 0.00 | 0.66 | 0.00 |
| AD5 | 1.70 | 0.61 | 0.00 | 0.82 | 1.92 | 0.00 | 0.35 | 0.93 | 1.02 | 0.33 | 0.49 | 2.14 |
| AD6 | 1.89 | 0.98 | 2.21 | 0.00 | 0.75 | 0.00 | 1.75 | 0.00 | 2.11 | 0.00 | 1.65 | 0.33 |
| AD7 | 0.26 | 0.00 | 0.00 | 3.15 | 0.00 | 2.67 | 0.00 | 0.00 | 0.00 | 0.19 | 1.92 | 2.26 |
| AD8 | 0.00 | 0.78 | 3.12 | 2.33 | 1.77 | 0.00 | 0.34 | 0.50 | 0.00 | 0.24 | 1.98 | 1.76 |
| AD9 | 0.49 | 0.71 | 0.33 | 0.00 | 0.25 | 2.39 | 1.98 | 0.00 | 0.36 | 0.99 | 1.07 | 0.26 |
| AD10 | 3.03 | 0.65 | 0.00 | 0.98 | 0.00 | 2.44 | 0.23 | 0.34 | 0.78 | 0.65 | 1.09 | 1.77 |
| AD11 | 0.39 | 2.98 | 0.33 | 0.00 | 1.28 | 0.77 | 0.00 | 1.76 | 1.98 | 0.00 | 0.54 | 0.00 |
| AD12 | 1.71 | 0.73 | 0.00 | 6.20 | 0.00 | 0.33 | 0.34 | 2.09 | 0.00 | 0.56 | 1.65 | 0.36 |
| AD13 | 0.25 | 0.00 | 0.00 | 0.00 | 0.00 | 1.3 | 9.5 | 0.00 | 2.10 | 0.26 | 1.70 | 0.03 |
| AD14 | 1.70 | 0.87 | 2.79 | 0.00 | 0.88 | 0.00 | 1.93 | 0.00 | 3.11 | 0.00 | 0.00 | 2.49 |
| AD15 | 1.41 | 5.34 | 0.00 | 0.00 | 0.31 | 0.00 | 3.31 | 0.00 | 1.71 | 0.21 | 0.16 | 0.00 |
| AD16 | 4.5 | 1.27 | 0.00 | 1.36 | 4.9 | 1.29 | 0.71 | 0.10 | 0.16 | 1.37 | 0.00 | 0.30 |
| CIS1 | 4.67 | 0.30 | 0.56 | 0.54 | 0.00 | 0.00 | 0.22 | 1.41 | 0.63 | 0.20 | 2.03 | 0.00 |
| CIS2 | 2.08 | 0.44 | 0.00 | 1.32 | 1.41 | 2.53 | 0.25 | 1.36 | 0.00 | 0.09 | 1.54 | 0.25 |
| CIS3 | 2.33 | 0.79 | 1.93 | 0.00 | 0.38 | 2.46 | 0.00 | 0.00 | 3.76 | 0.00 | 0.00 | 2.10 |
| CIS4 | 2.77 | 0.00 | 0.00 | 1.69 | 0.00 | 1.33 | 0.93 | 0.55 | 0.58 | 0.00 | 1.78 | 0.12 |
| CIS5 | 0.75 | 0.00 | 0.00 | 17.2 | 0.00 | 2.23 | 0.00 | 0.00 | 0.00 | 0.44 | 1.92 | 0.89 |
| CIS6 | 0.55 | 1.87 | 3.09 | 0.43 | 0.00 | 0.33 | 0.42 | 0.64 | 0.22 | 0.48 | 0.73 | 0.00 |
| CA1 | 0.88 | 0.00 | 0.00 | 3.71 | 0.38 | 2.82 | 0.70 | 0.20 | 0.29 | 2.11 | 0.00 | 0.00 |
| CA2 | 0.82 | 2.25 | 0.00 | 0.00 | 0.84 | 2.91 | 0.86 | 3.41 | 3.57 | 0.00 | 0.65 | 0.00 |
| CA3 | 0.39 | 0.00 | 3.11 | 0.55 | 0.00 | 3.09 | 0.00 | 0.71 | 3.15 | 0.61 | 2.88 | 0.73 |
| CA4 | 0.55 | 1.87 | 3.09 | 0.43 | 0.00 | 0.33 | 0.42 | 0.64 | 0.22 | 0.48 | 0.73 | 0.00 |
| CA5 | 0.00 | 1.42 | 0.60 | 0.71 | 0.00 | 0.00 | 0.32 | 1.52 | 0.71 | 0.10 | 2.21 | 0.00 |
| CA6 | 3.18 | 0.00 | 0.00 | 0.79 | 0.00 | 0.00 | 0.93 | 0.00 | 0.63 | 0.00 | 0.95 | 2.65 |
| CA7 | 1.71 | 0.77 | 0.00 | 0.00 | 0.00 | 0.32 | 25.4 | 2.45 | 0.00 | 0.78 | 0.10 | 0.30 |
| CA8 | 0.44 | 1.47 | 0.28 | 2.45 | 0.00 | 0.12 | 3.42 | 0.00 | 0.00 | 0.00 | 1.73 | 0.35 |
| CA9 | 0.44 | 15.1 | 0.00 | 0.00 | 0.00 | 1.55 | 0.00 | 0.00 | 0.00 | 0.25 | 2.03 | 0.34 |
| CA10 | 31.5 | 0.00 | 0.00 | 3.01 | 0.00 | 2.42 | 0.10 | 0.29 | 0.44 | 0.00 | 1.98 | 0.38 |
| CA11 | 0.45 | 0.00 | 0.00 | 27.2 | 0.00 | 3.23 | 0.00 | 0.00 | 0.00 | 0.84 | 2.92 | 0.89 |
| CA12 | 3.01 | 1.08 | 21.7 | 0.00 | 0.00 | 0.32 | 0.43 | 1.76 | 0.00 | 0.81 | 2.54 | 0.13 |
| CA13 | 26.1 | 0.00 | 0.00 | 2.31 | 0.00 | 1.99 | 0.87 | 0.23 | 0.32 | 0.00 | 1.74 | 0.65 |
| CA14 | 21.5 | 0.00 | 0.00 | 3.01 | 0.00 | 2.42 | 0.10 | 0.29 | 0.44 | 0.00 | 1.98 | 0.38 |
| CA15 | 3.44 | 0.67 | 0.71 | 0.00 | 0.32 | 1.90 | 2.31 | 0.13 | 0.77 | 0.16 | 0.89 | 0.91 |
| CA16 | 2.73 | 0.21 | 0.00 | 0.26 | 1.94 | 0.00 | 0.86 | 0.88 | 0.23 | 0.29 | 0.77 | 3.11 |
| CA17 | 0.00 | 2.21 | 1.33 | 0.71 | 0.00 | 0.66 | 0.92 | 1.69 | 3.56 | 2.16 | 0.00 | 0.77 |
| CA18 | 0.00 | 2.13 | 1.50 | 0.57 | 0.00 | 0.79 | 0.81 | 1.82 | 3.45 | 2.10 | 0.00 | 0.82 |
| CA19 | 2.81 | 0.00 | 1.71 | 26.2 | 0.00 | 0.00 | 1.88 | 0.00 | 0.50 | 0.00 | 0.41 | 0.55 |
| CA20 | 2.22 | 0.00 | 25.4 | 0.00 | 0.67 | 0.00 | 0.00 | 0.00 | 0.41 | 1.21 | 0.91 | 2.79 |
| CA21 | 2.12 | 0.00 | 35.4 | 0.00 | 0.77 | 0.00 | 0.33 | 0.00 | 0.40 | 0.56 | 0.81 | 2.79 |
| gene | p53 | p53 | p53 | p53 | p53 | p53 | p53 | p53 | p53 | p53 | p53 | TML |
| codon | 178 | 179 | 241 | 244 | 245 | 245 | 245 | 248 | 248 | 248 | 249 | |
| mutation | TGC | CCC | TTC | GCT | AGC | GCT | GAC | TGG | CAG | CTG | ATG | |
| N1 | 0.66 | 0.00 | 0.00 | 0.54 | 0.64 | 0.00 | 0.00 | 0.32 | 0.24 | 0.00 | 0.41 | 5.36 |
| N2 | 0.7 | 0.51 | 1.12 | 0.00 | 0.00 | 1.17 | 0.71 | 0.00 | 0.59 | 0.41 | 0.00 | 5.70 |
| N3 | 0.00 | 0.78 | 0.47 | 0.00 | 0.00 | 0.00 | 0.93 | 0.00 | 0.75 | 0.00 | 0.00 | 5.75 |
| N4 | 0.00 | 0.77 | 0.00 | 0.35 | 0.51 | 0.67 | 0.00 | 0.00 | 0.00 | 0.34 | 0.41 | 5.75 |
| N5 | 0.00 | 0.78 | 0.47 | 0.00 | 0.00 | 0.00 | 0.93 | 0.00 | 0.75 | 0.00 | 0.00 | 5.75 |
| N6 | 0.31 | 0.09 | 0.25 | 0.00 | 0.52 | 0.00 | 0.44 | 1.19 | 0.00 | 0.00 | 0.22 | 5.77 |
| N7 | 0.83 | 0.00 | 0.00 | 0.00 | 0.89 | 0.00 | 0.71 | 0.64 | 0.00 | 1.15 | 0.00 | 5.80 |
| N8 | 0.00 | 0.61 | 0.00 | 0.80 | 0.50 | 0.05 | 0.00 | 0.00 | 1.21 | 0.00 | 1.02 | 5.80 |
| N9 | 0.00 | 0.57 | 0.00 | 0.72 | 0.54 | 0.09 | 0.00 | 0.00 | 1.19 | 0.00 | 1.09 | 5.80 |
| N10 | 0.00 | 0.00 | 0.27 | 0.61 | 0.00 | 0.50 | 0.00 | 0.59 | 0.00 | 0.12 | 0.19 | 5.88 |
| N11 | 0.00 | 0.00 | 0.17 | 0.56 | 0.00 | 0.46 | 0.00 | 0.71 | 0.00 | 0.08 | 0.31 | 5.88 |
| N12 | 1.16 | 0.00 | 0.25 | 0.00 | 0.93 | 0.90 | 0.00 | 0.00 | 0.49 | 0.00 | 0.36 | 5.99 |
| N13 | 0.00 | 0.64 | 0.00 | 1.09 | 0.66 | 0.00 | 0.00 | 0.71 | 0.00 | 0.81 | 0.25 | 6.14 |
| N14 | 0.00 | 0.31 | 0.00 | 0.00 | 0.47 | 0.00 | 0.00 | 0.00 | 0.13 | 0.21 | 1.15 | 6.15 |
| N15 | 0.00 | 0.00 | 0.13 | 0.45 | 0.00 | 0.21 | 0.09 | 0.00 | 0.00 | 0.00 | 0.43 | 6.17 |
| N16 | 0.00 | 0.00 | 0.00 | 0.72 | 0.00 | 0.31 | 0.49 | 0.00 | 0.41 | 0.00 | 1.03 | 6.21 |
| N17 | 0.99 | 0.91 | 0.43 | 0.00 | 0.00 | 0.00 | 0.78 | 0.58 | 0.00 | 0.49 | 0.00 | 6.27 |
| N18 | 0.00 | 0.00 | 0.00 | 0.69 | 0.00 | 0.35 | 0.44 | 0.00 | 0.48 | 0.00 | 1.18 | 6.55 |
| N19 | 1.09 | 0.00 | 0.00 | 0.00 | 0.69 | 0.00 | 0.38 | 0.00 | 0.48 | 0.00 | 1.18 | 6.58 |
| N20 | 0.16 | 0.00 | 0.55 | 0.81 | 0.77 | 0.12 | 0.00 | 0.14 | 0.23 | 0.00 | 0.59 | 6.69 |
| N21 | 0.00 | 0.00 | 0.09 | 0.00 | 0.76 | 0.77 | 0.00 | 0.00 | 0.33 | 0.97 | 0.00 | 7.05 |
| N22 | 0.96 | 0.00 | 0.00 | 0.00 | 0.39 | 0.48 | 0.72 | 0.00 | 0.00 | 0.75 | 0.00 | 7.12 |
| N23 | 0.00 | 0.00 | 0.56 | 0.63 | 0.32 | 0.00 | 0.11 | 0.00 | 0.07 | 0.23 | 0.21 | 7.14 |
| N24 | 0.23 | 0.17 | 0.14 | 0.70 | 0.18 | 0.16 | 0.00 | 0.00 | 0.23 | 0.24 | 0.00 | 7.15 |
| AD1 | 0.00 | 0.51 | 0.00 | 0.68 | 0.57 | 0.12 | 3.44 | 0.00 | 1.13 | 0.00 | 1.99 | 16.50 |
| AD2 | 0.00 | 0.00 | 0.11 | 0.42 | 2.02 | 0.26 | 3.11 | 0.00 | 0.07 | 0.30 | 1.43 | 16.58 |
| AD3 | 2.21 | 0.21 | 0.94 | 0.15 | 0.07 | 0.30 | 0.00 | 0.77 | 0.00 | 2.21 | 0.19 | 16.84 |
| AD4 | 0.23 | 1.97 | 3.14 | 0.00 | 0.18 | 0.16 | 0.00 | 3.12 | 0.23 | 0.00 | 0.97 | 16.86 |
| AD5 | 0.00 | 0.00 | 0.21 | 0.16 | 1.99 | 2.03 | 0.00 | 0.22 | 0.15 | 0.00 | 2.35 | 17.42 |
| AD6 | 0.56 | 3.05 | 0.41 | 0.27 | 0.95 | 0.00 | 1.92 | 0.53 | 0.00 | 0.09 | 1.77 | 18.17 |
| AD7 | 0.73 | 3.42 | 0.77 | 0.65 | 0.00 | 0.51 | 0.00 | 0.00 | 0.31 | 0.00 | 0.28 | 18.42 |
| AD8 | 1.56 | 0.00 | 0.18 | 0.13 | 0.00 | 0.54 | 0.63 | 2.09 | 0.00 | 0.71 | 0.00 | 18.66 |
| AD9 | 0.77 | 0.25 | 0.66 | 0.00 | 2.41 | 2.02 | 1.01 | 0.00 | 0.00 | 2.11 | 0.71 | 18.77 |
| AD10 | 0.00 | 0.00 | 0.17 | 0.34 | 2.36 | 0.00 | 1.98 | 0.42 | 0.40 | 0.00 | 2.08 | 19.71 |
| AD11 | 0.00 | 0.35 | 1.32 | 0.55 | 0.78 | 1.88 | 2.08 | 0.37 | 0.00 | 0.41 | 1.99 | 19.76 |
| AD12 | 0.00 | 2.21 | 0.42 | 0.00 | 0.44 | 0.32 | 0.00 | 1.98 | 0.06 | 0.00 | 2.03 | 21.43 |
| AD13 | 0.16 | 1.50 | 0.00 | 0.71 | 0.00 | 0.40 | 2.21 | 0.00 | 1.31 | 0.27 | 0.00 | 21.7 |
| AD14 | 0.33 | 1.86 | 0.78 | 0.73 | 0.55 | 0.00 | 0.87 | 0.23 | 1.67 | 0.98 | 0.00 | 21.77 |
| AD15 | 0.25 | 1.87 | 0.00 | 2.02 | 0.08 | 0.00 | 2.44 | 0.22 | 2.36 | 0.12 | 0.20 | 22.01 |
| AD16 | 2.02 | 0.07 | 0.25 | 0.00 | 0.49 | 1.71 | 0.35 | 0.06 | 0.00 | 1.33 | 0.00 | 22.24 |
| CIS1 | 0.00 | 1.27 | 0.00 | 2.10 | 0.18 | 0.00 | 0.17 | 0.21 | 0.34 | 7.81 | 0.00 | 22.3 |
| CIS2 | 0.00 | 2.10 | 1.66 | 0.00 | 2.05 | 0.22 | 1.37 | 1.40 | 0.00 | 2.26 | 1.29 | 23.60 |
| CIS3 | 0.16 | 2.34 | 0.55 | 0.61 | 0.87 | 0.12 | 3.77 | 3.10 | 0.23 | 1.57 | 1.59 | 28.66 |
| CIS4 | 2.55 | 0.33 | 0.00 | 0.65 | 0.41 | 0.25 | 0.00 | 15.2 | 0.00 | 1.87 | 0.33 | 31.34 |
| CIS5 | 0.67 | 1.65 | 0.98 | 0.31 | 0.00 | 3.12 | 0.11 | 0.42 | 1.71 | 2.73 | 0.00 | 35.13 |
| CIS6 | 3.11 | 16.8 | 0.09 | 0.16 | 0.76 | 0.77 | 0.00 | 2.54 | 2.33 | 0.97 | 0.00 | 36.29 |
| CA1 | 3.07 | 0.66 | 0.75 | 0.91 | 0.49 | 2.87 | 0.00 | 0.44 | 0.67 | 3.30 | 0.81 | 25.06 |
| CA2 | 0.00 | 0.48 | 2.77 | 0.45 | 0.78 | 0.51 | 0.00 | 0.00 | 3.13 | 0.53 | 1.92 | 25.88 |
| CA3 | 0.00 | 0.00 | 0.98 | 0.91 | 0.00 | 3.92 | 0.71 | 0.49 | 3.83 | 0.00 | 0.00 | 26.06 |
| CA4 | 3.11 | 16.8 | 0.09 | 0.16 | 0.76 | 0.77 | 0.00 | 2.54 | 2.33 | 0.97 | 0.00 | 36.29 |
| CA5 | 0.00 | 1.33 | 0.00 | 2.34 | 0.28 | 0.00 | 0.21 | 0.19 | 0.43 | 25.41 | 0.00 | 37.78 |
| CA6 | 0.75 | 3.44 | 0.00 | 0.00 | 3.11 | 0.45 | 0.81 | 0.00 | 19.45 | 0.00 | 1.72 | 38.86 |
| CA7 | 1.23 | 1.45 | 0.45 | 0.19 | 0.69 | 0.00 | 1.91 | 0.04 | 0.54 | 1.52 | 2.16 | 42.01 |
| CA8 | 3.19 | 0.00 | 0.42 | 2.12 | 0.06 | 21.8 | 1.09 | 0.00 | 3.29 | 0.00 | 0.00 | 42.23 |
| CA9 | 0.71 | 1.95 | 16.5 | 0.25 | 0.00 | 0.22 | 0.00 | 0.00 | 0.42 | 1.31 | 1.78 | 42.85 |
| CA10 | 1.56 | 0.35 | 0.65 | 0.19 | 0.53 | 2.19 | 2.48 | 0.00 | 0.27 | 0.00 | 0.44 | 48.78 |
| CA11 | 0.77 | 2.65 | 0.78 | 0.91 | 0.00 | 2.12 | 0.11 | 0.42 | 3.71 | 2.73 | 0.00 | 49.73 |
| CA12 | 0.00 | 2.13 | 0.00 | 12.3 | 0.33 | 1.73 | 0.00 | 0.22 | 2.21 | 0.00 | 0.55 | 51.25 |
| CA13 | 1.44 | 0.76 | 0.15 | 0.24 | 0.34 | 0.00 | 13.7 | 0.00 | 0.00 | 0.00 | 0.56 | 51.40 |
| CA14 | 1.56 | 0.35 | 0.65 | 0.19 | 0.53 | 2.19 | 15.8 | 0.00 | 0.47 | 0.00 | 0.44 | 52.30 |
| CA15 | 0.65 | 0.58 | 2.21 | 2.54 | 1.64 | 0.00 | 3.02 | 0.00 | 0.24 | 30.8 | 0.00 | 53.89 |
| CA16 | 0.00 | 0.00 | 0.13 | 0.45 | 1.51 | 42.1 | 0.09 | 0.00 | 0.00 | 0.00 | 1.22 | 56.78 |
| CA17 | 0.00 | 3.72 | 0.18 | 0.24 | 35.69 | 0.53 | 1.46 | 1.66 | 0.00 | 0.00 | 3.20 | 60.69 |
| CA18 | 0.00 | 3.14 | 0.21 | 0.19 | 36.2 | 0.33 | 1.76 | 1.89 | 0.00 | 0.00 | 2.98 | 60.69 |
| CA19 | 0.79 | 1.98 | 0.00 | 1.40 | 0.00 | 0.56 | 0.32 | 0.00 | 26.79 | 0.00 | 0.00 | 65.90 |
| CA20 | 0.76 | 0.65 | 0.00 | 3.19 | 0.00 | 0.26 | 0.00 | 0.16 | 25.68 | 1.59 | 0.00 | 65.90 |
| CA21 | 0.66 | 2.98 | 0.00 | 3.25 | 0.00 | 0.36 | 0.00 | 0.70 | 14.9 | 1.60 | 0.00 | 67.63 |

TABLE S2

| gene | K-ras | K-ras | K-ras | K-ras | K-ras | K-ras | K-ras | K-ras | p53 | p53 | p53 | p53 |
|---|---|---|---|---|---|---|---|---|---|---|---|---|
| codon | 12 | 12 | 12 | 12 | 12 | 12 | 13 | 13 | 135 | 151 | 175 | 176 |
| mutation | GAT | GCT | AGT | GTT | CGT | TGT | GAC | TAC | CGC | TGC | CAT | CAC |
| N8 biopsy asc | 0.00 | 0.00 | 0.00 | 0.44 | 0.00 | 0.00 | 0.00 | 0.00 | 0.41 | 0.00 | 0.00 | 0.00 |
| N8 biopsy des | 0.00 | 0.00 | 0.00 | 0.00 | 0.00 | 0.00 | 0.00 | 0.00 | 0.00 | 0.21 | 0.70 | 0.11 |
| N8 biopsy trans | 0.00 | 0.00 | 0.00 | 0.00 | 0.23 | 0.00 | 0.00 | 0.00 | 0.00 | 0.26 | 0.67 | 0.00 |
| N8 biopsy pool | 0.00 | 0.00 | 0.00 | 0.57 | 0.34 | 0.00 | 0.00 | 0.00 | 0.50 | 0.29 | 0.79 | 0.00 |
| N8 stool | 0.00 | 0.00 | 0.00 | 0.00 | 0.00 | 0.00 | 0.00 | 0.00 | 0.28 | 0.16 | 0.83 | 0.00 |
| N13 biopsy asc | 0.00 | 0.31 | 0.00 | 0.00 | 0.00 | 0.00 | 0.00 | 0.22 | 0.00 | 0.23 | 0.00 | 0.55 |
| N13 biopsy des | 0.00 | 0.39 | 0.00 | 0.00 | 0.00 | 0.00 | 0.00 | 0.00 | 0.91 | 0.00 | 0.31 | 0.00 |
| N13 biopsy trans | 0.00 | 0.00 | 0.00 | 0.19 | 0.00 | 0.00 | 0.00 | 0.00 | 0.00 | 0.00 | 0.00 | 0.59 |
| N13 biopsy pool | 0.00 | 0.42 | 0.00 | 0.00 | 0.00 | 0.00 | 0.00 | 0.00 | 0.93 | 0.00 | 0.00 | 0.61 |
| N13 stool | 0.00 | 0.00 | 0.00 | 0.00 | 0.00 | 0.00 | 0.00 | 0.00 | 1.10 | 0.00 | 0.00 | 0.64 |
| N2 biopsy asc | 0.00 | 0.00 | 0.00 | 0.00 | 0.00 | 0.21 | 0.00 | 0.00 | 0.00 | 0.14 | 0.88 | 0.00 |
| N2 biopsy des | 0.16 | 0.00 | 0.33 | 0.00 | 0.00 | 0.00 | 0.00 | 0.00 | 0.00 | 0.00 | 0.00 | 0.44 |
| N2 biopsy trans | 0.00 | 0.00 | 0.40 | 0.00 | 0.00 | 0.00 | 0.00 | 0.00 | 0.00 | 0.00 | 0.80 | 0.60 |
| N2 biopsy pool | 0.00 | 0.00 | 0.42 | 0.00 | 0.00 | 0.00 | 0.00 | 0.00 | 0.00 | 0.00 | 0.91 | 0.61 |
| N2 stool | 0.00 | 0.00 | 0.00 | 0.00 | 0.00 | 0.00 | 0.00 | 0.00 | 0.00 | 0.00 | 0.70 | 0.51 |
| N12 biopsy asc | 0.00 | 0.00 | 0.00 | 0.00 | 0.24 | 0.00 | 0.00 | 0.00 | 0.24 | 0.89 | 0.90 | 0.00 |
| N12 biopsy des | 0.00 | 0.19 | 0.00 | 0.00 | 0.00 | 0.32 | 0.00 | 0.00 | 0.00 | 0.00 | 0.98 | 0.00 |
| N12 biopsy trans | 0.12 | 0.00 | 0.00 | 0.00 | 0.00 | 0.41 | 0.00 | 0.00 | 0.00 | 0.78 | 0.00 | 0.00 |
| N12 biopsy pool | 0.00 | 0.00 | 0.00 | 0.00 | 0.00 | 0.45 | 0.00 | 0.00 | 0.00 | 0.91 | 1.09 | 0.00 |
| N12 stool | 0.00 | 0.00 | 0.00 | 0.00 | 0.00 | 0.00 | 0.00 | 0.00 | 0.00 | 0.81 | 1.16 | 0.00 |
| gene | p53 | p53 | p53 | p53 | p53 | p53 | p53 | p53 | p53 | p53 | p53 | TML |
| codon | 178 | 179 | 241 | 244 | 245 | 245 | 245 | 248 | 248 | 248 | 249 | |
| mutation | TGC | CCC | TTC | GCT | AGC | GCT | GAC | TGG | CAG | CTG | ATG | |
| N8 biopsy asc | 0.45 | 0.00 | 0.22 | 0.00 | 0.79 | 0.00 | 0.00 | 0.49 | 0.00 | 0.00 | 0.00 | 2.80 |
| N8 biopsy des | 0.57 | 0.00 | 0.00 | 0.00 | 0.00 | 0.00 | 0.61 | 0.55 | 0.00 | 0.00 | 0.00 | 2.75 |
| N8 biopsy trans | 0.00 | 0.00 | 0.00 | 0.00 | 0.00 | 0.00 | 0.49 | 0.00 | 0.00 | 0.91 | 0.00 | 2.56 |
| N8 biopsy pool | 0.55 | 0.00 | 0.00 | 0.00 | 0.77 | 0.00 | 0.66 | 0.51 | 0.00 | 0.94 | 0.00 | 5.92 |
| N8 stool | 0.33 | 0.81 | 0.00 | 0.00 | 0.89 | 0.00 | 0.71 | 0.64 | 0.00 | 1.15 | 0.00 | 5.80 |
| N13 biopsy asc | 0.33 | 0.00 | 0.00 | 0.89 | 0.71 | 0.00 | 0.00 | 0.00 | 0.00 | 0.88 | 0.00 | 4.12 |
| N13 biopsy des | 0.29 | 0.00 | 0.00 | 0.00 | 0.77 | 0.00 | 0.00 | 0.55 | 0.00 | 0.00 | 0.00 | 3.22 |
| N13 biopsy trans | 0.00 | 0.00 | 0.00 | 0.96 | 0.00 | 0.00 | 0.00 | 0.61 | 0.00 | 0.93 | 0.20 | 2.88 |
| N13 biopsy pool | 0.00 | 0.00 | 0.00 | 0.99 | 0.81 | 0.00 | 0.00 | 0.69 | 0.00 | 0.96 | 0.00 | 5.41 |
| N13 stool | 0.88 | 0.00 | 0.00 | 1.09 | 0.66 | 0.00 | 0.00 | 0.71 | 0.00 | 0.81 | 0.25 | 6.14 |
| N2 biopsy asc | 0.00 | 0.31 | 0.99 | 0.00 | 0.00 | 1.10 | 0.82 | 0.00 | 0.00 | 0.49 | 0.25 | 5.19 |
| N2 biopsy des | 0.00 | 0.29 | 1.15 | 0.00 | 0.00 | 1.09 | 0.00 | 0.00 | 0.00 | 0.55 | 0.00 | 4.01 |
| N2 biopsy trans | 0.00 | 0.00 | 0.00 | 0.00 | 0.00 | 0.00 | 0.88 | 0.00 | 0.00 | 0.00 | 0.00 | 2.68 |
| N2 biopsy pool | 0.00 | 0.00 | 1.18 | 0.00 | 0.00 | 1.19 | 0.90 | 0.00 | 0.00 | 0.57 | 0.00 | 5.78 |
| N2 stool | 0.00 | 0.49 | 1.12 | 0.00 | 0.00 | 1.17 | 0.71 | 0.00 | 0.59 | 0.41 | 0.00 | 5.70 |
| N12 biopsy asc | 0.00 | 0.00 | 0.00 | 0.00 | 0.89 | 0.00 | 0.00 | 0.00 | 0.44 | 0.00 | 0.44 | 4.04 |
| N12 biopsy des | 0.33 | 0.00 | 0.29 | 0.00 | 0.00 | 0.78 | 0.00 | 0.00 | 0.56 | 0.00 | 0.00 | 3.45 |
| N12 biopsy trans | 0.00 | 0.00 | 0.33 | 0.00 | 0.00 | 0.00 | 0.00 | 0.00 | 0.31 | 0.13 | 0.00 | 1.96 |
| N12 biopsy pool | 0.00 | 0.00 | 0.35 | 0.00 | 0.99 | 0.88 | 0.00 | 0.00 | 0.51 | 0.21 | 0.50 | 5.89 |
| N12 stool | 1.09 | 0.00 | 0.25 | 0.00 | 0.93 | 0.90 | 0.00 | 0.00 | 0.49 | 0.00 | 0.36 | 5.99 |

Case with Normal Biopsies and Tumor;

TABLE S4

| gene | K-ras | K-ras | K-ras | K-ras | K-ras | K-ras | K-ras | K-ras | p53 | p53 | p53 | p53 |
|---|---|---|---|---|---|---|---|---|---|---|---|---|
| codon | 12 | 12 | 12 | 12 | 12 | 12 | 13 | 13 | 135 | 151 | 175 | 176 |
| mutation | GAT | GCT | GTT | AGT | CGT | TGT | GAC | TAC | CGC | TGC | CAT | CAC |
| AD2 stool | 2.81 | 0.23 | 0.00 | 0.25 | 2.12 | 0.00 | 0.15 | 0.41 | 0.09 | 1.91 | 0.00 | 0.00 |
| AD2 biopsy | 2.47 | 0.28 | 0.00 | 0.00 | 2.32 | 0.00 | 0.22 | 0.52 | 0.15 | 2.02 | 0.00 | 0.00 |
| AD3 stool | 0.55 | 0.34 | 1.65 | 0.91 | 0.00 | 2.07 | 0.25 | 0.88 | 1.5 | 0.00 | 2.21 | 0.21 |
| AD3 biopsy | 0.00 | 0.51 | 1.83 | 1.16 | 0.00 | 2.15 | 0.53 | 0.99 | 1.75 | 0.00 | 2.52 | 0.00 |
| AD5 stool | 1.70 | 0.61 | 0.00 | 0.82 | 1.92 | 0.00 | 0.35 | 0.93 | 0.49 | 2.14 | 0.00 | 0.00 |
| AD5 biopsy | 0.00 | 0.82 | 0.00 | 1.03 | 2.11 | 0.00 | 0.43 | 0.99 | 0.61 | 2.32 | 0.00 | 0.00 |
| AD6 stool | 1.89 | 0.98 | 2.21 | 0.00 | 0.75 | 0.00 | 1.75 | 0.00 | 1.65 | 0.33 | 0.56 | 3.05 |
| AD6 biopsy | 2.10 | 1.05 | 2.56 | 0.00 | 0.87 | 0.00 | 1.86 | 0.00 | 1.95 | 0.46 | 0.61 | 0.00 |
| AD7 stool | 0.26 | 0.00 | 0.00 | 3.15 | 0.00 | 2.67 | 0.00 | 0.00 | 1.92 | 2.26 | 0.73 | 3.42 |
| AD7 biopsy | 0.31 | 0.00 | 0.00 | 3.30 | 0.00 | 2.85 | 0.00 | 0.00 | 2.03 | 2.45 | 0.93 | 3.61 |
| AD8 stool | 0.00 | 0.78 | 3.12 | 2.33 | 1.77 | 0.00 | 0.34 | 0.50 | 1.98 | 1.76 | 1.56 | 0.00 |
| AD8 biopsy | 0.00 | 0.98 | 3.33 | 2.67 | 1.96 | 0.00 | 0.52 | 0.65 | 2.17 | 1.96 | 1.74 | 0.00 |
| AD9 stool | 0.49 | 0.71 | 0.33 | 0.00 | 0.25 | 2.39 | 1.98 | 0.00 | 1.07 | 0.26 | 0.77 | 0.25 |
| AD9 biopsy | 0.61 | 0.94 | 0.00 | 0.00 | 0.51 | 2.66 | 2.12 | 0.00 | 1.18 | 0.35 | 0.98 | 0.34 |
| AD10 stool | 3.03 | 0.65 | 0.00 | 0.98 | 0.00 | 2.44 | 0.23 | 0.34 | 1.09 | 1.77 | 0.00 | 0.00 |
| AD10 biopsy | 3.34 | 0.87 | 0.00 | 1.15 | 0.00 | 2.68 | 0.54 | 0.00 | 1.17 | 2.01 | 0.00 | 0.00 |
| AD11 stool | 0.39 | 2.98 | 0.33 | 0.00 | 1.28 | 0.77 | 0.00 | 1.76 | 0.54 | 0.00 | 0.00 | 0.35 |
| AD11 biopsy | 0.48 | 3.11 | 0.51 | 0.00 | 1.43 | 0.98 | 0.00 | 0.00 | 0.71 | 0.00 | 0.00 | 0.52 |
| AD12 stool | 1.71 | 0.73 | 0.00 | 6.20 | 0.00 | 0.33 | 0.34 | 2.09 | 1.65 | 0.36 | 0.00 | 2.21 |
| AD12 biopsy | 1.93 | 0.99 | 0.00 | 6.59 | 0.00 | 0.54 | 0.00 | 2.13 | 1.88 | 0.51 | 0.00 | 2.43 |
| AD13 stool | 0.25 | 0.00 | 0.00 | 0.00 | 0.00 | 1.3 | 9.5 | 0.00 | 1.70 | 0.03 | 0.16 | 1.50 |
| AD13 biopsy | 0.33 | 0.00 | 0.00 | 0.00 | 0.00 | 1.62 | 10.1 | 0.00 | 1.98 | 0.14 | 0.22 | 1.65 |
| AD14 stool | 1.70 | 0.87 | 2.79 | 0.00 | 0.88 | 0.00 | 1.93 | 0.00 | 0.00 | 2.49 | 0.33 | 1.86 |
| AD14 biopsy | 0.00 | 0.87 | 2.79 | 0.00 | 0.88 | 0.00 | 1.93 | 0.00 | 0.00 | 2.49 | 0.33 | 1.86 |
| AD15 stool | 1.41 | 5.34 | 0.00 | 0.00 | 0.31 | 0.00 | 3.31 | 0.00 | 0.16 | 0.00 | 0.25 | 1.87 |
| AD15 biopsy | 1.62 | 6.10 | 0.00 | 0.00 | 0.54 | 0.00 | 3.61 | 0.00 | 0.22 | 0.00 | 0.37 | 1.99 |
| AD16 stool | 4.5 | 1.27 | 0.00 | 1.36 | 4.9 | 1.29 | 0.71 | 0.10 | 0.00 | 0.30 | 2.02 | 0.07 |
| AD16 biopsy | 5.10 | 1.45 | 0.00 | 0.00 | 5.23 | 1.41 | 0.98 | 0.21 | 0.00 | 0.54 | 2.19 | 0.18 |
| CIS1 stool | 4.67 | 0.30 | 0.56 | 0.54 | 0.00 | 0.00 | 0.22 | 1.41 | 2.03 | 0.00 | 0.00 | 1.27 |
| CIS1 biopsy | 5.20 | 0.47 | 0.71 | 0.00 | 0.00 | 0.00 | 0.39 | 1.62 | 2.24 | 0.00 | 0.00 | 1.43 |
| CIS2 stool | 2.08 | 0.44 | 0.00 | 1.32 | 1.41 | 2.53 | 0.25 | 1.36 | 1.54 | 0.25 | 0.00 | 2.10 |
| CIS2 biopsy | 2.34 | 0.61 | 0.00 | 0.00 | 1.67 | 2.77 | 0.52 | 1.54 | 1.78 | 0.33 | 0.00 | 2.32 |
| CIS3 stool | 2.33 | 0.79 | 1.93 | 0.00 | 0.38 | 2.46 | 0.00 | 0.00 | 0.00 | 2.10 | 0.16 | 2.34 |
| CIS3 biopsy | 2.41 | 0.98 | 2.08 | 0.00 | 0.50 | 2.59 | 0.00 | 0.00 | 0.00 | 2.35 | 0.28 | 2.64 |
| CIS4 stool | 2.77 | 0.00 | 0.00 | 1.69 | 0.00 | 1.33 | 0.93 | 0.55 | 1.78 | 0.12 | 2.55 | 0.33 |
| CIS4 biopsy | 2.92 | 0.00 | 0.00 | 1.98 | 0.00 | 1.52 | 1.14 | 0.71 | 1.91 | 0.23 | 2.74 | 0.00 |
| CIS5 stool | 0.75 | 0.00 | 0.00 | 17.2 | 0.00 | 2.23 | 0.00 | 0.00 | 1.92 | 0.89 | 0.67 | 1.65 |
| CIS5 biopsy | 0.87 | 0.00 | 0.00 | 19.6 | 0.00 | 2.44 | 0.00 | 0.00 | 2.12 | 1.09 | 0.71 | 1.73 |
| CIS6 stool | 0.55 | 1.87 | 3.09 | 0.43 | 0.00 | 0.33 | 0.42 | 0.64 | 0.73 | 0.00 | 3.11 | 16.8 |
| CIS6 biopsy | 0.70 | 2.01 | 3.16 | 0.51 | 0.00 | 0.56 | 0.49 | 0.00 | 0.98 | 0.00 | 3.31 | 17.9 |
| CA7 stool | 1.71 | 0.77 | 0.00 | 0.00 | 0.00 | 0.32 | 25.4 | 2.45 | 0.10 | 0.30 | 1.23 | 1.45 |
| CA7 biopsy | 2.01 | 0.92 | 0.00 | 0.00 | 0.00 | 0.47 | 27.6 | 2.33 | 0.00 | 0.53 | 1.11 | 1.60 |
| CA10 stool | 31.5 | 0.00 | 0.00 | 3.01 | 0.00 | 2.42 | 0.10 | 0.29 | 1.98 | 0.38 | 1.56 | 0.35 |
| CA10 biopsy | 33.2 | 0.00 | 0.00 | 3.13 | 0.00 | 2.51 | 0.22 | 0.39 | 2.12 | 0.46 | 1.78 | 0.44 |
| CA13 stool | 26.1 | 0.00 | 0.00 | 2.31 | 0.00 | 1.99 | 0.87 | 0.23 | 1.74 | 0.65 | 1.44 | 0.76 |
| CA13 biopsy | 28.2 | 0.00 | 0.00 | 2.55 | 0.00 | 2.13 | 0.97 | 0.42 | 1.88 | 0.79 | 1.53 | 0.00 |
| CA15 stool | 3.44 | 0.67 | 0.71 | 0.00 | 0.32 | 1.90 | 2.31 | 0.13 | 0.89 | 0.91 | 0.65 | 0.58 |
| CA15 biopsy | 3.61 | 0.51 | 0.92 | 0.00 | 0.50 | 2.11 | 2.45 | 0.26 | 1.07 | 1.15 | 0.77 | 0.71 |
| CA20 stool | 2.22 | 0.00 | 25.4 | 0.00 | 0.67 | 0.00 | 0.00 | 0.00 | 0.91 | 2.79 | 0.76 | 1.98 |
| CA20 biopsy | 2.39 | 0.00 | 27.2 | 0.00 | 0.88 | 0.00 | 0.00 | 0.00 | 1.09 | 2.93 | 0.83 | 2.21 |
| gene | p53 | p53 | p53 | p53 | p53 | p53 | p53 | p53 | p53 | p53 | p53 | TML |
| codon | 178 | 179 | 241 | 244 | 245 | 245 | 245 | 248 | 248 | 248 | 249 | |
| mutation | TGC | CCC | TTC | GCT | AGC | GCT | GAC | TGG | CAG | CTG | ATG | |
| AD2 stool | 0.33 | 0.56 | 0.11 | 0.42 | 2.02 | 0.26 | 3.11 | 0.00 | 0.07 | 0.30 | 1.43 | 16.58 |
| AD2 biopsy | 0.33 | 0.00 | 0.21 | 0.51 | 2.11 | 0.31 | 3.22 | 0.00 | 0.12 | 0.41 | 1.51 | 16.71 |
| AD3 stool | 0.93 | 0.71 | 0.94 | 0.15 | 0.07 | 0.30 | 0.00 | 0.77 | 0.00 | 2.21 | 0.19 | 16.84 |
| AD3 biopsy | 1.09 | 0.91 | 1.18 | 0.30 | 0.00 | 0.55 | 0.00 | 0.91 | 0.00 | 2.43 | 0.35 | 19.16 |
| AD5 stool | 1.02 | 0.33 | 0.21 | 0.16 | 1.99 | 2.03 | 0.00 | 0.22 | 0.15 | 0.00 | 2.35 | 17.42 |
| AD5 biopsy | 1.17 | 0.55 | 0.40 | 0.00 | 2.20 | 2.13 | 0.00 | 0.37 | 0.27 | 0.00 | 2.67 | 18.07 |
| AD6 stool | 2.11 | 0.00 | 0.41 | 0.27 | 0.95 | 0.00 | 1.92 | 0.53 | 0.00 | 0.09 | 1.77 | 18.17 |
| AD6 biopsy | 2.17 | 0.00 | 0.40 | 0.00 | 1.09 | 0.00 | 2.17 | 0.73 | 0.00 | 0.13 | 1.97 | 20.12 |
| AD7 stool | 0.00 | 0.19 | 0.77 | 0.65 | 0.00 | 0.51 | 0.00 | 0.00 | 0.31 | 0.00 | 0.28 | 18.42 |
| AD7 biopsy | 0.00 | 0.00 | 0.90 | 0.77 | 0.00 | 0.72 | 0.00 | 0.00 | 0.55 | 1.65 | 0.00 | 20.07 |
| AD8 stool | 0.00 | 0.24 | 0.18 | 0.13 | 0.00 | 0.54 | 0.63 | 2.09 | 0.00 | 0.71 | 0.00 | 18.66 |
| AD8 biopsy | 0.00 | 0.39 | 0.28 | 0.26 | 0.00 | 0.63 | 0.88 | 2.17 | 0.00 | 1.01 | 0.00 | 21.60 |
| AD9 stool | 0.36 | 0.99 | 0.66 | 0.00 | 2.41 | 2.02 | 1.01 | 0.00 | 0.00 | 2.11 | 0.71 | 18.77 |
| AD9 biopsy | 0.00 | 0.00 | 0.77 | 0.00 | 2.62 | 2.19 | 1.19 | 0.00 | 0.00 | 2.32 | 0.97 | 19.75 |
| AD10 stool | 0.78 | 0.65 | 0.17 | 0.34 | 2.36 | 0.00 | 1.98 | 0.42 | 0.40 | 0.00 | 2.08 | 19.71 |
| AD10 biopsy | 0.00 | 0.78 | 0.24 | 0.60 | 2.51 | 0.00 | 2.11 | 0.69 | 0.67 | 0.00 | 2.17 | 21.53 |
| AD11 stool | 1.98 | 0.00 | 1.32 | 0.55 | 0.78 | 1.88 | 2.08 | 0.37 | 0.00 | 0.41 | 1.99 | 19.76 |
| AD11 biopsy | 2.14 | 0.00 | 1.56 | 0.76 | 0.00 | 2.09 | 2.23 | 0.60 | 0.00 | 0.00 | 2.16 | 19.28 |
| AD12 stool | 0.00 | 0.56 | 0.42 | 0.00 | 0.44 | 0.32 | 0.00 | 1.98 | 0.06 | 0.00 | 2.03 | 21.43 |
| AD12 biopsy | 0.00 | 0.66 | 0.00 | 0.00 | 0.65 | 0.42 | 0.00 | 2.19 | 0.18 | 0.00 | 2.15 | 23.25 |
| AD13 stool | 2.10 | 0.26 | 0.00 | 0.71 | 0.00 | 0.40 | 2.21 | 0.00 | 1.31 | 0.27 | 0.00 | 21.70 |
| AD13 biopsy | 2.34 | 0.35 | 0.00 | 0.95 | 0.00 | 0.61 | 2.32 | 0.00 | 1.44 | 0.40 | 0.00 | 24.45 |
| AD14 stool | 3.11 | 0.00 | 0.78 | 0.73 | 0.55 | 0.00 | 0.87 | 0.23 | 1.67 | 0.98 | 0.00 | 21.77 |
| AD14 biopsy | 3.11 | 0.00 | 0.78 | 0.73 | 0.00 | 0.00 | 0.87 | 0.23 | 1.67 | 0.00 | 0.00 | 18.54 |
| AD15 stool | 1.71 | 0.21 | 0.00 | 2.02 | 0.08 | 0.00 | 2.44 | 0.22 | 2.36 | 0.12 | 0.20 | 22.01 |
| AD15 biopsy | 1.97 | 0.00 | 0.00 | 2.13 | 0.19 | 0.00 | 2.61 | 0.50 | 2.47 | 0.00 | 0.41 | 24.74 |
| AD16 stool | 0.16 | 1.37 | 0.25 | 0.00 | 0.49 | 1.71 | 0.35 | 0.06 | 0.00 | 1.33 | 0.00 | 22.24 |
| AD16 biopsy | 0.25 | 1.46 | 0.39 | 0.00 | 0.62 | 1.90 | 0.00 | 0.17 | 0.00 | 1.57 | 0.00 | 23.65 |
| CIS1 stool | 0.63 | 0.20 | 0.00 | 2.10 | 0.18 | 0.00 | 0.17 | 0.21 | 0.34 | 7.81 | 0.00 | 22.3 |
| CIS1 biopsy | 0.87 | 0.44 | 0.00 | 2.26 | 0.00 | 0.00 | 0.26 | 0.33 | 0.50 | 8.02 | 0.00 | 24.74 |
| CIS2 stool | 0.00 | 0.09 | 1.66 | 0.00 | 2.05 | 0.22 | 1.37 | 1.40 | 0.00 | 2.26 | 1.29 | 23.60 |
| CIS2 biopsy | 0.00 | 0.21 | 1.89 | 0.00 | 2.24 | 0.00 | 1.51 | 1.62 | 0.00 | 2.33 | 1.41 | 25.09 |
| CIS3 stool | 3.76 | 0.00 | 0.55 | 0.61 | 0.87 | 0.12 | 3.77 | 3.10 | 0.23 | 1.57 | 1.59 | 28.66 |
| CIS3 biopsy | 0.00 | 0.00 | 0.68 | 0.79 | 0.96 | 0.30 | 3.91 | 3.32 | 0.31 | 1.62 | 0.00 | 25.72 |
| CIS4 stool | 0.58 | 0.00 | 0.00 | 0.65 | 0.41 | 0.25 | 0.00 | 15.2 | 0.00 | 1.87 | 0.33 | 31.34 |
| CIS4 biopsy | 0.67 | 0.00 | 0.00 | 0.79 | 0.55 | 0.38 | 0.00 | 17.1 | 0.00 | 2.11 | 0.00 | 34.75 |
| CIS5 stool | 0.00 | 0.44 | 0.98 | 0.31 | 0.00 | 3.12 | 0.11 | 0.42 | 1.71 | 2.73 | 0.00 | 35.13 |
| CIS5 biopsy | 0.00 | 0.46 | 1.17 | 0.50 | 0.00 | 3.35 | 0.21 | 0.55 | 1.92 | 2.88 | 0.00 | 39.6 |
| CIS6 stool | 0.22 | 0.48 | 0.09 | 0.16 | 0.76 | 0.77 | 0.00 | 2.54 | 2.33 | 0.97 | 0.00 | 36.29 |
| CIS6 biopsy | 0.11 | 0.23 | 0.16 | 0.25 | 0.00 | 0.95 | 0.00 | 2.73 | 2.49 | 1.09 | 0.00 | 37.63 |
| CA7 stool | 0.00 | 0.78 | 0.45 | 0.19 | 0.69 | 0.00 | 1.91 | 0.04 | 0.54 | 1.52 | 2.16 | 42.01 |
| CA7 biopsy | 0.00 | 0.89 | 0.00 | 0.11 | 0.78 | 0.00 | 2.15 | 0.10 | 0.76 | 1.70 | 2.09 | 45.15 |
| CA10 stool | 0.44 | 0.00 | 0.65 | 0.19 | 0.53 | 2.19 | 2.48 | 0.00 | 0.27 | 0.00 | 0.44 | 48.78 |
| CA10 biopsy | 0.52 | 0.00 | 0.71 | 0.33 | 0.70 | 2.30 | 2.58 | 0.00 | 0.35 | 0.00 | 0.59 | 52.33 |
| CA13 stool | 0.32 | 0.00 | 0.15 | 0.24 | 0.34 | 0.00 | 13.7 | 0.00 | 0.00 | 0.00 | 0.56 | 51.40 |
| CA13 biopsy | 0.47 | 0.00 | 0.22 | 0.00 | 0.50 | 0.00 | 16.3 | 0.00 | 0.00 | 0.00 | 0.73 | 56.70 |
| CA15 stool | 0.77 | 0.16 | 2.21 | 2.54 | 1.64 | 0.00 | 3.02 | 0.00 | 0.24 | 30.8 | 0.00 | 53.89 |
| CA15 biopsy | 0.98 | 0.30 | 2.39 | 2.67 | 1.99 | 0.00 | 3.31 | 0.00 | 0.54 | 33.1 | 0.00 | 59.34 |
| CA20 stool | 0.41 | 0.66 | 0.00 | 3.19 | 0.00 | 0.26 | 0.00 | 0.16 | 24.9 | 1.59 | 0.00 | 65.90 |
| CA20 biopsy | 0.00 | 0.87 | 0.00 | 0.00 | 0.00 | 0.37 | 0.00 | 0.29 | 26.3 | 1.77 | 0.00 | 67.13 |

1. A method of evaluating the risk of cancer development in a subject, comprising the steps of:
(1) providing from said subject a test sample of material for which said risk of cancer development is to be evaluated;
(2) quantitating the frequency of one or more mutated alleles in said test sample, relative to one or more nonmutated alleles; and
(3) comparing said frequency of said one or more mutated alleles in said test sample with a reference frequency, wherein a higher frequency of said one or more mutated alleles in said test sample than in said reference frequency indicates that said subject has an elevated risk of cancer,
thereby evaluating the risk of cancer development in said subject.

2. The method of claim 1, wherein said one or more mutated alleles are obtained from a cancer-associated gene.

3. The method of claim 1, wherein said one or more mutated alleles are selected from the group consisting of alleles of K-ras and of p53.

4. The method of claim 1, wherein the frequency of 15 or more mutated alleles is quantitated.

5. The method of claim 1, wherein the frequency of 20 or more mutated alleles is quantitated.

6. The method of claim 1, wherein said cancer is selected from the group consisting of adenoma, carcinoma in situ, and invasive carcinoma.

7. The method of claim 1, wherein said reference frequency is derived from one or more references subject that do not have cancer.

8. The method of claim 1, wherein said test sample is selected from the group consisting of blood, urine, a tumor biopsy, a tumor aspirate, a cultured tumor cell, bone marrow, a stool sample, a cathartic preparation and a colonic brushing.

9. A method of evaluating the risk of colorectal cancer development in a subject, comprising the steps of:
(1) providing from said subject a test sample of material for which said risk of colorectal cancer development is to be evaluated;
(2) quantitating the frequency of one or more mutated alleles in said test sample, relative to one or more nonmutated alleles; and
(3) comparing said frequency of said one or more mutated alleles in said test sample with a reference frequency, wherein a higher frequency of said one or more mutated alleles in said test sample than in said reference frequency indicates that said subject has an elevated risk of colorectal cancer,
thereby evaluating the risk of colorectal cancer development in said subject.

10. The method of claim 9, wherein said cancer is selected from the group consisting of adenoma, carcinoma in situ and invasive carcinoma.

11. The method of claim 9, wherein said test sample comprises an exfoliated cell.

12. The method of claim 9, wherein said test sample is selected from the group consisting of a colonic lavage, a stool sample, and a colonic brushing.

13. The method of claim 9, wherein the frequency of said allele is below about 1.2% and the subject does not have colorectal cancer.

14. The method of claim 9, wherein the frequency of said allele is between about 1.2% and about 9.5% and the subject has adenoma or carcinoma in situ.

15. The method of claim 9, wherein the frequency of said allele is above about 9.5% and the subject has invasive carcinoma.

16. The method of claim 9, wherein said reference frequency is derived from one or more reference subject that do not have colorectal cancer.

17. The method of claim 9, wherein the step of quantitating the frequency of one or more mutated alleles in said test sample is performed using a oligonucleotide array.

18. The method of claim 9, wherein the step of quantitating further comprises enhancement of signal using rolling circle amplification.

19. A method of evaluating the risk of pancreatic cancer development in a subject, comprising the steps of:
(1) providing from said subject a test sample of material for which said risk of pancreatic cancer development is to be evaluated;
(2) quantitating the frequency of one or more mutated alleles in said test sample, relative to one or more nonmutated alleles; and
(3) comparing said frequency of said one or more mutated alleles in said test sample with a reference frequency, wherein a higher frequency of said one or more mutated alleles in said test sample than in said reference frequency indicates that said subject has an elevated risk of pancreatic cancer,
thereby evaluating the risk of pancreatic cancer development in said subject.

20. The method of claim 19, wherein said cancer is selected from the group consisting of pre-cancerous pancreatitis and carcinoma.

21. The method of claim 19, wherein said test sample comprises pancreatic juice obtained by canulation of the pancreatic duct or after stimulation with secretin.

22. The method of claim 19, wherein the frequency of said allele is below about 1.2% and the subject does not have pancreatic cancer.

23. The method of claim 19, wherein the frequency of said allele is between about 1.2% and about 3.8% and the subject has precancerous pancreatitis.

24. The method of claim 19, wherein the frequency of said allele is above about 3.8% and the subject has pancreatic cancer.

25. A method of evaluating the stage of cancer development in a subject, comprising the steps of:
(1) providing from said subject a test sample of material for which said stage of cancer development is to be evaluated;
(2) quantitating the frequency of one or more mutated alleles in said test sample; and
(3) comparing said frequency of one or more mutated alleles in said test sample with the frequency of one or more reference alleles, wherein a mutated allele in higher frequency than a reference allele indicates that said subject has a cancer of a given stage.

26. The method of claim 25, wherein said one or more mutated alleles are obtained from a cancer-associated gene.

27. The method of claim 25, wherein said one or more mutated alleles are selected from the group consisting of alleles of K-ras and of p53.

28. The method of claim 25, wherein said cancer is a colorectal cancer selected from the group consisting of adenoma, carcinoma in situ and invasive carcinoma.

29. The method of claim 25, wherein the frequency of said allele is below about 1.2% and the subject does not have colorectal cancer.

30. The method of claim 25, wherein the frequency of said allele is between about 1.2% and about 9.5% and the subject has adenoma or carcinoma in situ.

31. The method of claim 25, wherein the frequency of said allele is above about 9.5% and the subject has invasive carcinoma.

32. A method of diagnosis of colorectal cancer in a subject, comprising the steps of:
(1) providing from said subject a test sample of material, wherein said test sample comprises one or more cells or cellular material;
(2) determining the frequency of mutated alleles of one or more genes in said test sample, wherein said one or more genes are selected from the group consisting of K-ras, p53, APC, and BAT26;
(3) quantitating the mutational load in said test sample, wherein said mutational load comprises the sum of the frequencies determined in step (2); and
(4) comparing said mutational load in said test sample with a reference mutational load, wherein a higher mutational load in said test sample than in said reference frequency indicates that said subject has colorectal cancer.

33. The method of claim 32, wherein the mutational load of said test sample is below about 6.2% and the subject does not have colorectal cancer.

34. The method of claim 32, wherein the mutational load of said test sample is between about 6.2% and about 22.2% and the subject has adenoma.

35. The method of claim 32, wherein the mutational load of said test sample is between about 22.3% and about 36.3% and the subject has carcinoma in situ.

36. The method of claim 32, wherein the mutational load of said test sample is above about 25.1% and the subject has subject has invasive carcinoma.

37. A method of evaluating the likelihood of relapse of cancer in a subject suffering therefrom, comprising the steps of:
(1) providing from said subject a first sample and a second sample of material, wherein said second sample is provided from said subject a sufficient period of time after said first sample;
(2) quantitating the frequency of one or more mutated alleles relative to one or more nonmutated alleles in said first and second samples; and
(3) comparing said frequency of said first sample with said frequency from said second sample, wherein a higher frequency in said second sample than in said first sample indicates that said subject has an elevated risk of relapse of cancer.

38. The method of claim 37, wherein said first sample is provided before a cancer treatment is administered to the subject and wherein said second sample is provided after a cancer treatment is administered to the subject.

39. The method of claim 37, wherein said one or more mutated alleles are obtained from a cancer-associated gene.

40. The method of claim 37, wherein said one or more mutated alleles are selected from the group consisting of alleles of K-ras and of p53.

41. The method of claim 37, wherein the frequency of 15 or more mutated alleles is quantitated.

42. The method of claim 37, wherein said cancer is a colorectal cancer selected from the group consisting of adenoma, carcinoma in situ and invasive carcinoma.

43. The method of claim 37, wherein said first sample is selected from the group consisting of a colonic lavage, a stool sample, and a colonic brushing.

44. A method of determining the predisposition to a relapsing colorectal cancer of a given stage in a subject, comprising the steps of:
(1) providing from said subject a first sample and a second sample of material, wherein said second sample is provided from said subject a sufficient period of time after said first sample;
(2) quantitating the frequency of one or more mutated alleles in said first and second samples; and
(3) comparing said frequency of one or more mutated alleles in said first sample with the frequency of one or more alleles in said second sample, wherein a mutated allele in higher frequency in said first sample than said allele in said second sample indicates that said subject is predisposed to a relapsing colorectal cancer of a given stage.

45. The method of claim 44, wherein said colorectal cancer is selected from the group consisting of adenoma, carcinoma in situ and invasive carcinoma.

46. The method of claim 44, wherein the frequency of said allele in said second sample is below about 1.2% and the subject does not have relapsing colorectal cancer.

47. The method of claim 44, wherein the frequency of said allele in said second sample is between about 1.2% and about 9.5% and the subject has adenoma or carcinoma in situ.

48. The method of claim 44, wherein the frequency of said allele in said second sample is above about 9.5% and the subject has invasive carcinoma.

49. A population of nucleic acid molecules comprising a first nucleic acid molecule and a second nucleic acid molecule, wherein said first and second nucleic acid molecules each comprise a mutated allele obtained from a gene selected from a cancer-associated gene.

50. The population of claim 49, wherein said one or more mutated alleles are selected from the group consisting of alleles of K-ras and of p53.

51. The population of claim 49, wherein said one or more mutated alleles are selected from the group consisting of the alleles listed in Table 1.

52. The population of claim 49, wherein said nucleic acid molecules are covalently bound to a solid or semi-solid support medium.

53. The population of claim 49, wherein said solid or semi-solid support medium comprises an array.

54. The population of claim 49, further comprising a means for detecting said mutated alleles.

55. A kit comprising a population of nucleic acid molecules containing a first nucleic acid molecule and a second nucleic acid molecule wherein said first and second nucleic acid molecules each comprise a mutated allele obtained from a gene selected from the group consisting of K-ras, p53, APC, and BAT26, means for obtaining from a subject a test sample, and instructions for use thereof.

56. The kit of claim 55, further comprising a means for calculating the frequency of mutated alleles in a sample from the subject or the total mutational load of the sample.
